(19) United States
(12) Patent Application Publication
(10) Pub. No.: US 2023/0235348 A1
(43) Pub. Date: Jul. 27, 2023

(54) CONTROL OF MEIOTIC CROSSOVER IN MAIZE

(71) Applicants: Cold Spring Harbor Laboratory, Cold Spring Harbor, NY (US); Cambridge Enterprise Limited, Cambridge (GB)

(72) Inventors: Robert Martienssen, Cold Spring Harbor, NY (US); Charles Underwood, Cold Spring Harbor, NY (US); Ian Henderson, Cambridge (GB)

(73) Assignees: Cold Spring Harbor Laboratory, Cold Spring Harbor, NY (US); Cambridge Enterprise Limited, Cambridge (GB)

(21) Appl. No.: 17/813,577

(22) Filed: Jul. 19, 2022

Related U.S. Application Data
(62) Division of application No. 16/311,439, filed on Dec. 19, 2018, now abandoned.
(60) Provisional application No. 62/356,957, filed on Jun. 30, 2016.

(51) Int. Cl. C12N 15/82; A01H 1/02; C12N 15/113

(52) U.S. Cl. CPC C12N 15/8261; A01H 1/02; C12N 15/113; C12N 15/8218; Y02A 40/146

(57) ABSTRACT

The present disclosure provides methods for increasing meiotic recombination in crop plants, as well as plants and seeds produced by such methods.

### RELATED APPLICATIONS

This application is a divisional under 35 U.S.C. § 120 of U.S. Application Serial No. 16/311,439, filed Dec. 19, 2018, which is a national stage filing under 35 U.S.C. § 371 of International Application No. PCT/US2017/039919, filed Jun. 29, 2017, which was published under PCT Article 21(2) in English and claims the benefit under 35 U.S.C. § 119(e) of U.S. Provisional Application No. 62/356,957, filed Jun. 30, 2016, each of which is herein incorporated by reference in its entirety.

### GOVERNMENT FUNDING

This invention was made with government support under IOS-1025830 awarded by National Science Foundation and under 2R01GM067014-11 awarded by National Institutes of Health. The government has certain rights in the invention.

### REFERENCE TO AN ELECTRONIC SEQUENCE LISTING

The contents of the electronic sequence listing (C130070031US02-SEQ-DQB.xml; Size: 57, 244 bytes; and Date of Creation: Jul. 19, 2022) is herein incorporated by reference in its entirety.

### BACKGROUND

Maize (Zea mays) is a major cereal crop, and millions of people living in the tropical and subtropical zones of the world are largely dependent on maize for their subsistence. Maize C4 crop that can maintain high rates of photosynthetic activity, which is important for grain yield and biomass (Pathi KM et al. Plant Signal Behav. 2013; 8(10):e25891). Being a cross-pollinating species, it maintains broad morphological features, genetic variability and geographical adaptability. Among the cereals, maize is the most important crop in the world in terms of productivity, industrial products (fermentation and pharmaceuticals), animal feed and fodder. Maize yield is largely affected by various biotic and abiotic stresses. Several factors such as lack of useful variation and the long time duration required in conventional breeding affect the development of plants resistant to biotic and abiotic stresses through conventional breeding.

Meiotic crossover brings together alleles of genes that control traits and is fundamental to the success and speed of plant breeding, by allowing the introduction of new traits from distant relatives into elite varieties. A major barrier to breeding in crop plants, such as maize, is suppression of recombination in pericentromeric heterochromatin, which can occupy 80% of the chromosome, effectively preventing incorporation of new traits controlled by genes in or near these regions of the chromosome.

### SUMMARY

In genetics, the phenomenon of “linkage” is the co-inheritance of genetic traits that are physically located on the same chromosome. Meiotic recombination, during sexual reproduction, has the potential to split up linked traits by physically recombining homologous chromosomes. Related to this, in crop breeding “linkage drag” refers to the process by which genetic traits that are desirable to separate are instead co-inherited due to their location in chromosomal regions suppressed for meiotic recombination. Provided herein are data that show that “linkage drag” in hybrid plants that are useful for breeding purposes is reduced by down regulating an epigenetic pathway that functions in flowering plants (the “H3K9me2 and non-CG DNA methylation pathway”). Reducing “linkage drag” permits the production of novel genetic combinations, which are not naturally produced. This will facilitate the exploration of many genetic combinations and make it possible to produce novel plant phenotypes, including improved resistance to abiotic and biotic stresses.

Production of a hybrid plant results from crossing one plant variety to another plant variety. Meiotic recombination between chromosomes from the different parental lines in the hybrid plant results in the segregation of alleles and development of new traits. Meiotic recombination is typically restricted to gene-rich regions, usually found at the ends of chromosomes and is suppressed in gene-sparse heterochromatic regions in the center of chromosomes. In Arabidopsis, for example, recombination is suppressed in heterochromatin. In some crop plants, such as maize and wheat, these recombination-suppressed regions can cover up to 80% or 90% of the chromosome.

In plants, DNA methylation occurs in three sequence contexts, CG, CHG and CHH (where H is either an A, T or C nucleotide ). CHG methylation is maintained by specific enzymes: in Arabidopsis, primarily by Chromomethylase 3 (CMT3); and in corn, primarily by CMT3 ortholog Zea mays methyltransferase 2 (ZMET2) and Zea mays methyltransferase 5 (ZMET5).

The effects of mutants in the histone H3 lysine 9 methylation (H3K9me2) and non-CG DNA methylation pathway in Arabidopsis on meiotic recombination have been assessed. Results indicate that mutations affecting the CHG methylation pathway can reduce recombination suppression in heterochromatin through their effect on CHG methylation. Progressive reduction of DNA methylation at CHG methylation sites in recombination-suppressed heterochromatin led to corresponding increases in recombination in that heterochromatin. By reducing the extent of CHG methylation, one can increase the meiotic recombination rate in heterochromatin as well as in transposon-rich regions that are heavily modified by CHG methylation.

In contrast, loss of CG methylation in Arabidopsis leads to increased suppression of heterochromatic recombination. The data described herein, therefore, illustrate that decreasing the extent of different types of DNA methylation (CHG vs CG) has distinct, opposite effects on the rate of meiotic recombination in heterochromatin.

In an analogous fashion, specific mutants have been observed to have different, distinct effects on meiotic recombination when introduced into inbred lines versus hybrid lines. For example, although fancm mutants successfully boost meiotic crossovers (Crismani W, et al. Science 2012; 336(6088): 1588-90), this boost in meiotic crossovers did not translate into hybrid Arabidopsis lines (Girard C, et al. PLoS Genet. 2015; 11(7): e1005369). Herein, results show that CEN3 crossover frequency significantly increased in cmt3 inbreds and, surprisingly, also in F₁ hybrids carrying a cmt3 mutation (FIG. 1B).

Thus, provided herein are compositions and methods for producing hybrid plants, such as hybrid corn plants, for use as intermediates in plant breeding for the purpose of introducing new traits into a plant variety.

Also provided herein are compositions and methods for increasing meiotic recombination rate in a recombination-suppressed chromosomal region in a plant, such as a corn plant, including a hybrid corn plant. For example, such hybrid corn plants may comprise homologous chromosomes carrying genetic polymorphisms, some of which encode useful new traits, but are located within chromosomal regions that are recombination suppressed. The compositions and methods described herein for increasing meiotic recombination events in these recombination-suppressed chromosomal regions provide for hybrid plants that are useful as intermediates in the process of plant breeding.

Further provided herein are hybrid corn plants that have been modified to increase meiotic recombination events in a recombination suppressed region. As an example, such modified hybrid corn plants may comprise an expression cassette that functions as a dominant negative suppressor (e.g., amiRNA, co-suppression cassette, antisense RNA, or virus induced gene silencing vector) of ZMET2, ZMET5 or KRYPTONITE, which are homologs of the Arabidopsis chromomethylases (ZMET2 and ZMET5) and histone H3K9 methyltransferases (KRYPTONITE).

### BRIEF DESCRIPTION OF THE DRAWINGS

FIGS. 1A-1G show genome-wide increases in pericentromeric crossover frequency in cmt3 mutants. FIG. 1A, Crossing scheme: Col chromosomes are black and Ler chromosomes are blue. FIG. 1B, Heterochromatic CEN3 crossover frequency (cM) in wildtype and cmt3, in Col/Col inbreds, or Col/Ler F1 hybrids. Replicate measurements are shown in black and mean values in red. FIG. 1C, Euchromatic 420 crossover frequency in wildtype and cmt3, with Col/Col homozygosity, or Col/Ler heterozygosity, shown as for FIG. 1B. FIG. 1D, Crossover frequency measured in wildtype (blue) and cmt3 (red) F2 populations. Mean values are indicated by the horizontal dotted lines and centromeres by the vertical dotted lines. The heterochromatic knob inversion on the short arm of chromosome 4 is indicated by the black box and ‘INV’. FIG. 1E, Crossovers from wildtype and cmt3 (black) plotted as in FIG. 1D, overlaid with published BS-seq data (Stroud H, et al. Cell 2013; 152: 352-64) shown in CG (red), CHG (blue) or CHH (green) sequence contexts. FIG. 1F, Distribution of crossovers per F2 individual for wildtype and cmt3 populations. Red dotted lines indicate mean values. FIG. 1G, Normalized crossover distributions analyzed along chromosome telomere (TEL) to centromere (CEN) axes in wildtype (blue) or cmt3 (red) populations.

FIG. 2 shows a flow diagram of a timeline of a maize transformation protocol (Ishida Y et al. Nature Protocols 2007: 2: 1614-21).

FIGS. 3A-3C show schematics of the protocols described in Example 3.

FIGS. 4A-4B show schematics of the protocols described in Example 7.

### DESCRIPTION

The centromeres of eukaryotic chromosomes are surrounded by repetitive heterochromatic regions, which are suppressed for both meiotic crossover and gene transcription. In fission yeast, histone 3 lysine 9 dimethylation (H3K9me2) is guided to heterochromatin by RNA interference (RNAi), and genetic ablation of the pathway induces mitotic homologous recombination and meiotic crossover in pericentromeric heterochromatin. In addition, plants and mammals silence heterochromatin by DNA cytosine methylation, which can occur in CG and non-CG (CHG and CHH) sequence contexts (where H=A, C or T). Data provided herein shows that the downregulation of the interlinked H3K9me2 and non-CG DNA methylation pathway in Arabidopsis increases meiotic crossover within pericentromeric heterochromatin. Reduction of these epigenetic marks causes increases in pericentromeric crossover frequency. The present disclosure provides results showing that activation of recombination occurs in both inbred and hybrid backgrounds. Suppression of pericentromeric recombination severely limits plant breeding. As described herein, mutation of the H3K9me2 and non-CG DNA methylation pathway presents an attractive solution to unlock genetic diversity in hybrid crops, such as hybrid maize (corn).

Provided herein, in some embodiments, are F₁ hybrid maize plants and seeds that include an expression cassette that functions as a dominant negative suppressor of a gene in the H3K9me2 and non-CG DNA methylation pathway. In some embodiments, F₁ hybrid maize plants and seeds include an expression cassette that includes a maize promoter that is active during meiosis and is operably linked to a nucleic acid encoding an amiRNA that targets a gene in the H3K9me2 and non-CG DNA methylation pathway. In some embodiments, F₁ hybrid maize plants and seeds include an expression cassette that includes a maize promoter that is active during meiosis and is operably linked to a nucleic acid encoding an antisense RNA that targets a gene in the H3K9me2 and non-CG DNA methylation pathway. In some embodiments, F₁ hybrid maize plants and seeds include an expression cassette that includes a maize promoter that is active during meiosis and is operably linked to a nucleic acid encoding virus induced gene silencing vector. In some embodiments, F₁ hybrid maize plants and seeds include an expression cassette that includes a maize promoter that is active during meiosis and is operably linked to a nucleic acid encoding long double stranded RNA (which are processed into small RNA molecules).

Also provided herein are F₁ hybrid maize seeds produced by crossing two different maize lines (e.g., maize inbred lines), one of which comprises expression cassette that functions as a dominant negative suppressor of a gene in the H3K9me2 and non-CG DNA methylation pathway (e.g., an expression cassette that includes a maize promoter that is active during meiosis and is operably linked to a nucleic acid encoding an amiRNA that targets a gene in the H3K9me2 and non-CG DNA methylation pathway).

The “H3K9me2 and non-CG DNA methylation pathway,” as discussed above, is an epigenetic pathway in flowering plants that silences gene expression and meiotic recombination. Genes of the maize H3K9me2 and non-CG DNA methylation pathway that may be targeted, as provided herein, include, but are not limited to, homologs of the Arabidopsis chromomethylases (e.g., CMT1, CMT2 and CMT3), such as ZMET2, ZMET5 and homologs of the Arabidopsis histone H3K9 methyltransferases, such as KRYPTONITE. Other genes of the maize H3K9me2 and non-CG DNA methylation pathway may also be targeted, alone or in combination with ZMET2, ZMET5 and KRYPTONITE.

An “inbred” plant is a plant having a genome that is genetically homozygous (there are few genetic polymorphisms—for example three or less, including no-genetic polymorphisms between homologous chromosomes). Inbred plants are usually produced by multiple generations of self-pollination. If an individual plant is heterozygous at a locus, approximately seven generations of self-pollination are needed until nearly all the progeny generated from that single plant are homozygous. After seven consecutive generations of self-pollination, more than 99% of the progeny are expected to be homozygous at any one locus. This process of repeated self-pollinations is known as inbreeding.

An “F₁ hybrid” plant is a first-generation plant produced by crossing two genotypically different plants (e.g., plants from different inbred lines). The genome of an F₁ hybrid plant is genetically heterozygous (there are many, for example more than three, genetic polymorphisms between the homologous chromosomes).

An “F₂” plant is a second-generation plant produced by inbreeding an F₁ hybrid plant. By comparison, a “BC₁ hybrid” plant is a second-generation plant produced by crossing an F₁ hybrid plant to a parental plant or a plant from a parental line (a line used to produce the F₁ hybrid plant).

Inbred parental plants, in some embodiments, carry an expression cassette that comprises a maize promoter that is active during meiosis. An “expression cassette” refers to a nucleic acid (e.g., DNA) that comprises a promoter operably linked to a sequence of nucleotides encoding a molecule of interest (e.g., RNA or protein). A “promoter” is a control region of a nucleic acid at which initiation and rate of transcription of the remainder of a nucleic acid sequence are controlled. A promoter is considered to be “operably linked” when it is in a correct functional location and orientation relative to a sequence of nucleic acid that it regulates (e.g., to control (“drive”) transcriptional initiation and/or expression of that sequence). Expression cassettes of the present disclosure, in some embodiments, use “maize promoters,” which are promoters active in maize plants. Maize promoters (maize promoter sequences) for use as provided herein may be derived in whole or in part from a maize plant, a plant other than maize, or a combination thereof (a recombinant maize promoter), provided the promoter is active in maize plants. A maize promoter that is “active during meiosis” refers to a maize promoter that controls transcriptional initiation and/or expression of a downstream sequence during any one or more stages of maize meiosis. A maize promoter that is active during meiosis may also be active during other stages of development. By comparison, a maize promoter that is “specifically active” during meiosis refers to a maize promoter that is active only during meiosis, or is active primarily during meiosis. Examples of such “meiosis-specific” maize promoters include, but are not limited to, AMEIOTIC1 (GRMZM5G883855; Pawlowski WP, et al. PNAS 2008; 106(9): 3603-08), ABSENCE OF FIRST DIVISION (AFD1/REC8) (GRMZM2G059037; Golubovskaya IN, et al. Journal of Cell Science 2006; 119: 3306-3315), and DMC1 (GRMZM2G109618; Etedali F, International journal of Agronomy and Plant Production 2012; 3(3): 100-108), and PHS1 (GRMZM2G100103; Ronceret A, et al. PNAS 2009; 106(47): 20121-26). Other meiosis-specific maize promoters are encompassed by the present disclosure.

Gene silencing nucleic acids, generally, are nucleic acids that are capable of reducing expression of genes during meiosis, during gene transcription or during post-transcriptional processes. Well-known examples of approaches by which gene silencing can be carried out include RNA interference (RNAi) and RNA silencing. RNAi, for example, leads to post transcriptional gene silencing triggered by double-stranded RNA molecules. Other examples of gene silencing molecules for use in plants include antisense RNA, nucleic acids encoding virus induced gene silencing (VIGS) vectors, and long double stranded RNA, which are processed into small RNA molecules.

“Artificial microRNAs (amiRNAs)” are small RNAs that can be genetically engineered and function to specifically silence single or multiple genes of interest (Tiwari M, et al. Plant Mol. Biol. 2014; 86(1-2): 1-18). An amiRNA is complementary to and binds to a target RNA (e.g., mRNA), the binding of which elicits a reduction (e.g., 20%-100%, 30%-100%, 40%-100%, 50%-100%, 60%-100%, 70%-100%, 20%-95%, 30%-95%, 40%-95%, 50%-95%, 60%-95%, 70%-95%, 20%-90%, 30%-90%, 40%-90%, 50%-90%, 60%-90% or 70%-90% reduction) in expression of the target RNA. The length of an amiRNA may vary. In some embodiments, an amiRNA has a length of 15-50 nucleotides, or 15-30 nucleotides. For example, an amiRNA may have a length of 15, 16, 17, 18, 19, 20, 21, 22, 23, 24, 25, 26, 27, 28, 29 or 30 nucleotides. In some embodiments, an amiRNA has a length of 21 nucleotides. Examples of amiRNA targets are provided as SEQ ID NO: 1-10 (targeting maize ZMET2), SEQ ID NO: 11-20 (targeting maize ZMET5), and SEQ ID NO: 21-30 (targeting maize KRYPTONITE).

An amiRNA may be expressed, for example, as part of a backbone having a sequence based on the maize microRNA gene zma-miR396h (Meng X, et al. The Plant Cell 2011; 23(3): 942-60). The amiRNA sequence may replace the endogenous miR396 sequence. As another example, the amiRNA may be expressed as part of a backbone based on a rice microRNA gene Oryza sativa MIR528 (osa-miR528) that has been modified for amiRNA production (Yan F, et al. J of Biotech. 2012; 160(3-4): 146-50).

“Virus-induced gene silencing (VIGS)” refers to sequence-specific reduction in target gene expression by infection of a plant with a virus vector containing fragments of the target gene (see, e.g., Ruiz MT et al. Plant Cell 1998; 10(6): 937-46; and Baulcombe DC, Current Opinion in Plant Biology 1999; 2(2): 109-13, each of which is incorporated by reference).

A “V₀” plant is a plant infected with virus.

A “V₁” plant is a progeny plant of a virus-infected V₀ plant.

Also provided herein are methods of producing maize plants that carry (e.g., chromosomally or episomally) an expression cassette that has a maize promoter that is active during meiosis and operably linked to a nucleic acid encoding an amiRNA (or other dominant negative suppressor, such as an antisense RNA or virus induced gene silencing vector) that targets a gene in the H3K9me2 and non-CG DNA methylation pathway. The methods may comprise, for example, (a) introducing into (transforming into) an embryo of a maize inbred plant or a cell of a maize inbred plant an expression cassette that includes a maize promoter that is active during meiosis and is operably linked to a nucleic acid encoding an amiRNA that targets ZMET2, ZMET5 or KRYPTONITE to produce an embryo comprising the expression cassette or a cell comprising the expression cassette, (b) regenerating the embryo comprising the expression cassette or the cell comprising the expression cassette to produce a T₀ maize plant, and (c) crossing the T₀ maize plant to another inbred plant to produce maize seed (e.g., F₁ hybrid seed).

Methods of transforming plants and plant parts with nucleic acids are known, any of which may be used to introduce an expression cassette into a plant or plant part, as provided herein. Examples include, but are not limited to agrobacterium-mediated transformation of maize embryos (Gelvin SB Microbiol Mol Biol Rev. 2003; 67(1): 16-37) followed by plant regeneration (Ishida Y et al. Nature Protocols 2007: 2: 1614-21), by particle bombardment (Kikkert JR et al. Methods Mol Biol. 2005; 286:61-78) or similar approaches, such as PEG-mediated protoplast transformation, silicon carbide whisker, or electroporation (see, e.g., Que Q, et al. Front Plant Sci 2014; 5: 379).

A “T₀ plant” is a plant regenerated following transformation of plant embryos/cells with a nucleic acid construct. A plant may be regenerated, for example, from immature embryos (Green CE, Philips RL. Crop Sci. 1975;15:417-21; Duncan DR, et al. Planta. 1985;165:322-32; Bohorova NE, et al. Maydica. 1995;40:275-81; Ishida Y, et al. Nat Biotechnol. 1996; 14:745-50; and Aguado-Santacruz GA, et al. In Vitro Cell Dev Biol Plant. 2007;43:215-24), mature embryos (Huang XQ, Wei ZM. Plant Cell Rep. 2004;22:793-800; and Al-Abed D, et al. Planta. 2006;223:1355-60), nodal regions (Sidorov V, et al. Plant Cell Rep. 2006;25:320-8), leaf tissues (Conger BV, et al. Plant Cell Rep. 1987;6:345-7; and Ahmadabadi M, et al. Transgenic Res. 2007;16:437-48), anthers (Ting YC, et al. Plant Sci Lett. 1981;23:139-45; and Barloy D, Beckert M. Plant Cell Tissue Org. Cult. 1993;33:45-50), tassel and ear meristems (Pareddy DR, Petolino JF. Plant Sci. 1990;46:225-32), protoplast (Morocz C, et al. Theor Appl Genet. 1990;80:721-6), and shoot meristems (Sairam RV, I. 2003;46:323-9).

For example, maize callus from embryo scutellar tissues may be initiated and maintained on MS medium inorganic components, Straus medium vitamins and amino acids, 20 g sucrose and 8 g agar per liter, and 2 mg 2,4-dichlorophenoxyacetic acid (2,4-D)/liter. Callus may be maintained in subculture every 21 to 28 days and remained capable of differentiation for 9 months. Regeneration of complete plants may be accomplished by subculture of callus to 0.25 mg 2,4-D/liter for 30 days followed by transfer to 2,4-D-free culture medium. At 0.25 mg 2,4-D/liter numerous curled and wrinkled leaves typically develop. Complete plants differentiate after transfer to 2,4-D-free medium. After transplantation to soil, plants survive and grow normally. The optimum embryo age for scutellar callus initiation is, in some embodiments, 18 days post-pollination. Hormone combinations such as 1 mg 2,4-D, 4 mg a-naphthaleneacetic acid (NAA), and 0.05 mg 6-(γ,γ-dimethyl allylamino)-purine (2iP)/liter may increase the efficiency of scutellar callus initiation (Green CE, Philips RL. Crop Sci. 1975;15:417-21).

Maize seeds (e.g., F1 hybrid seeds) may be grown to produce F₁ maize plants. In some embodiments, an F₁ maize plant (comprising the amiRNA expression cassette) is backcrossed to a plant from a parental line or other line to produce BC₁ maize seed. The backcross breeding method is typically used to incorporate specific traits into elite lines. This method works by crossing a transgenic inbred line (e.g., carrying the amiRNA expression cassette) with an elite inbred line. The offspring of this cross, referred to as the F₁ hybrid generation, should have 50% of the transgenic line’s alleles and 50% of the elite line’s alleles. An “elite” maize inbred line generally refers to a maize line that has many alleles that confer many strong agronomic traits resulting in high yields in a particular environment. An “exotic” maize inbred line refers to a non-elite line that contains a particular favorable trait, e.g., drought tolerance, but otherwise has poor characteristics compared to elite lines. Exotic lines can be a useful source of novel genetic variants that can be introgressed into elite lines to confer the useful trait. The F₁ plants are crossed back to the elite inbred again. These offspring are referred to as the BC₁ generation (backcross 1). The BC₁ offspring may be genotyped by extracting DNA and performing an amplification assay (e.g., PCR using cassette-specific primers) on the extracted DNA to determine which of the BC₁ offspring are transgenic (e.g., which of the offspring carry the amiRNA expression cassette).

In some embodiments, an F₁ maize plant is self-pollinated to produce F₂ maize seed. Under self-pollination, the silks of an ear are pollinated by pollen from the same plant.

The BC₁ maize seed, or F₂ maize seed, may then be grown to produce a BC₁ maize plant, or an F₂ maize plant, respectively. The mature plants may then be screened for desirable traits. Examples of such desirable traits include, but are not limited to, herbicide tolerance, insecticide tolerance, increased starch, insect resistance, disease resistance, and drought tolerance, salt tolerance, waterlogging tolerance and soil pH tolerance.

Epigenetic drift in plants could affect crossover distribution over evolutionary time, reminiscent of genetic drift in mammalian systems. Suppression of recombination in repetitive regions is a major barrier to the introduction of genetic diversity, causing significant linkage drag in elite maize and wheat varieties, where most of the chromosome is composed of pericentromeric heterochromatin. H3K9me2 and non-CG DNA methylation mutants should unlock pericentromeric crossover in crop breeding programmes.

### EXAMPLES

### Example 1. Epigenetic Control of Meiotic Crossover in Arabidopsis Pericentromeric Heterochromatin

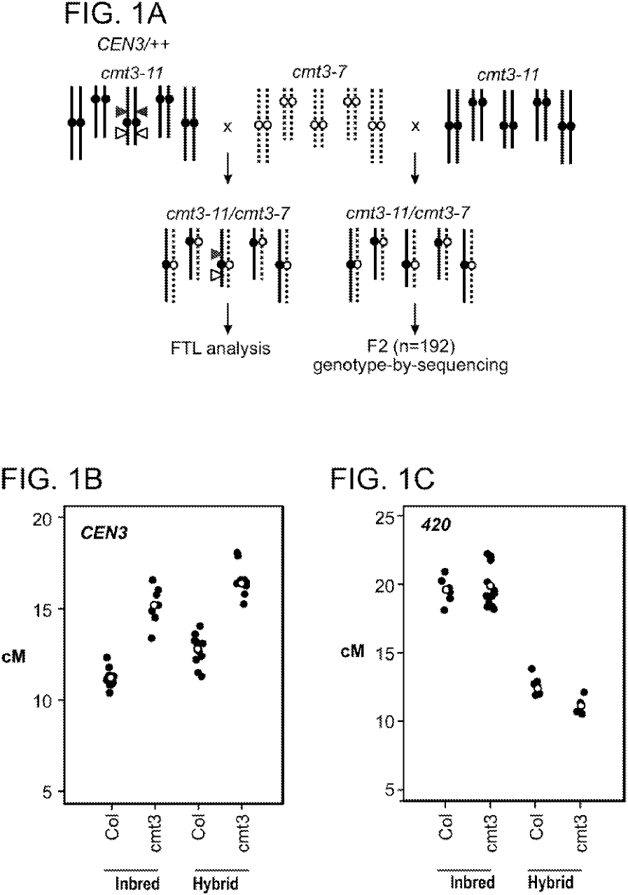

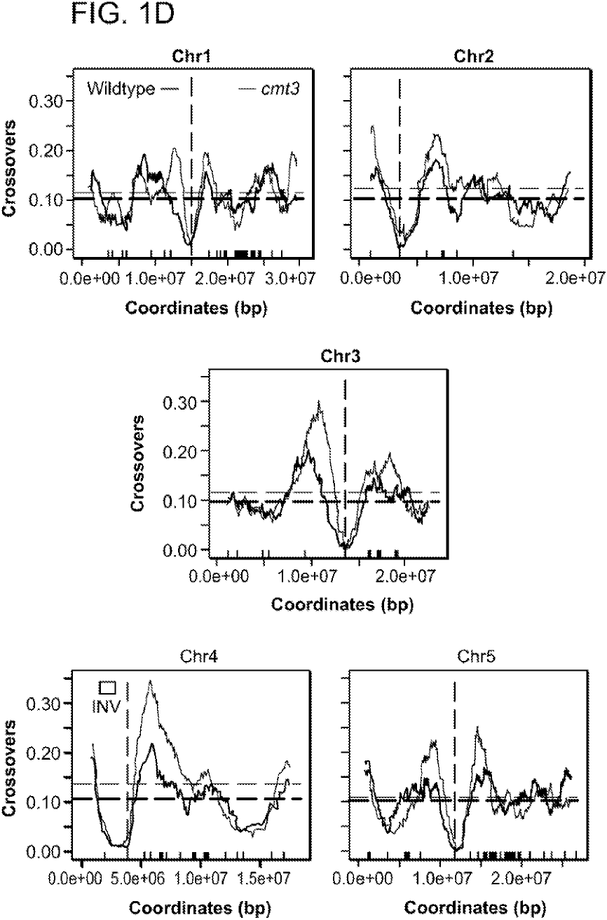

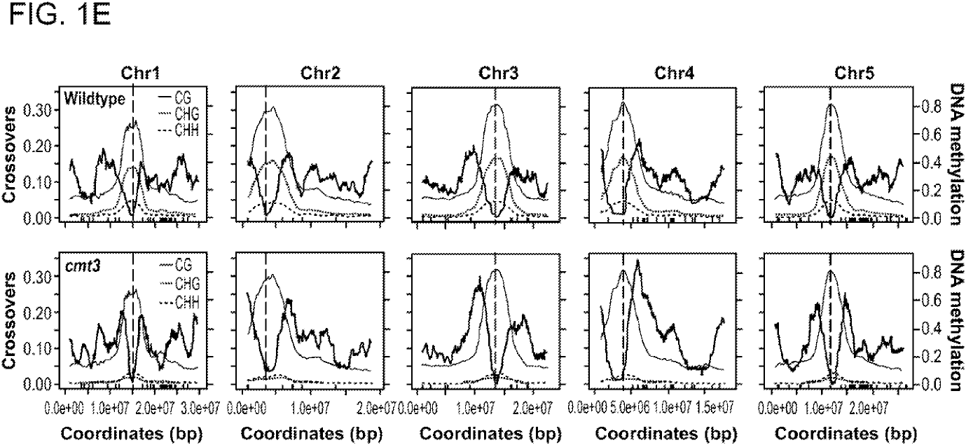

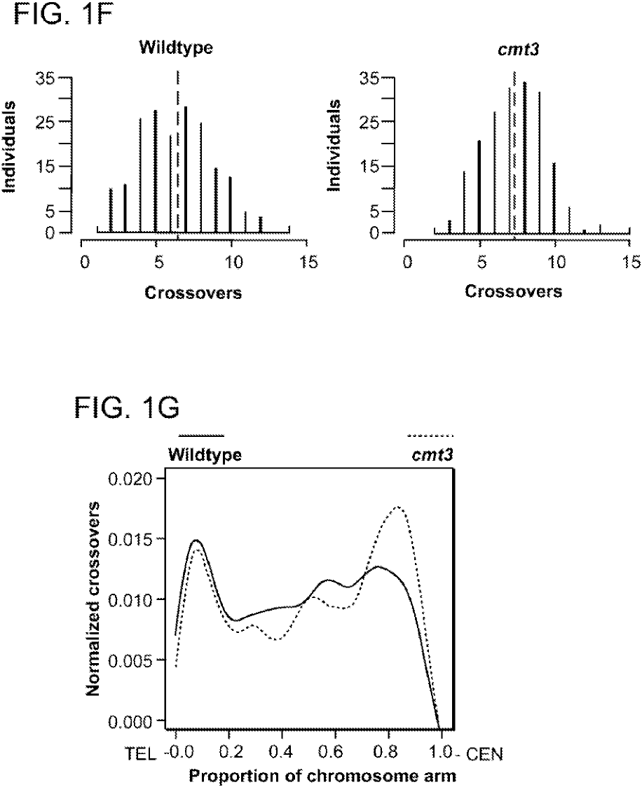

To measure recombination frequency H3K9me2 and DNA methylation mutants were combined with fluorescent crossover reporter lines (Fluorescent Tagged Lines, FTLs) (Berchowitz LE, Copenhaver GP. Nat. Protoc. 2008; 3: 41-50). In order to test whether pericentromeric crossovers were increased in hybrid backgrounds, the CEN3 FTL pollen line with cmt3 alleles in Col (cmt3-11) and Ler (cmt3-7) accessions were used (Lindroth AM, et al. Science 2001; 292: 2077-80) (FIG. 1A). CEN3 crossover frequency significantly increased in both cmt3 inbreds (X² P=3.00×10⁻¹⁴³) and, unexpectedly, in F₁ hybrids (X² P=1.27×10⁻⁸⁶) (FIG. 1B). In contrast, the euchromatic 420 FTL interval on chromosome 3 did not significantly change in cmt3 inbreds (X² P=0.651), and slightly decreased in hybrids (X² P= 1.96×10⁻³) (FIG. 1C). Crossovers genome-wide were then mapped by generating large wildtype and cmt3 F₂ populations (FIG. 1A). In order to map crossover distributions genome-wide at high resolution, genotyping-by-sequencing was performed on 192 F₂ individuals from wildtype and cmt3 populations (Yelina NE, et al. Genes Dev. 2015; 29: 2183-202) (FIGS. 1D, 1E, 1F and 1G). The mean number of crossovers per wildtype F₂ individual (mean=6.41) was comparable to that observed in similar populations (Giraut L, et al. PLoS Genet. 2011; 7: e1002354), and a significant increase in total crossovers in the cmt3 population was observed (mean=7.34) (Mann-Whitney-Wilcoxon test P=8.86×10⁻⁵) (FIGS. 1D, 1E).

Crossover distributions were analysed according to their location in the euchromatic arms, heterochromatic pericentromeres and centromeres (FIGS. 1D, 1E). Centromeres were defined as the regions that surround centromeric genome sequence assembly gaps (version TAIR10) that fail to undergo recombination in wildtype (Copenhaver G P, et al. Science 1999; 286: 2468-74; and Giraut L, et al. PLoS Genet. 2011; 7: e1002354). Pericentromeric heterochromatin was defined as regions flanking the centromeres with higher than average DNA methylation, and the euchromatic arms were defined as the remainder of the chromosomes. Crossovers were significantly increased in cmt3 pericentromeres (X² P=6.50×10⁻⁵), which are strongly depleted of CHG DNA methylation (FIGS. 1D-1F). We also observed centromeric crossovers in cmt3 but not wildtype controls (18 versus 0) (X² P= 1.85×10⁻⁴), but crossovers were still suppressed in centromeres, either because of residual non-CG DNA methylation and H3K9me2, other chromatin modifications, or structural polymorphisms (Ito H, et al. Genet. Genomics 2007; 277: 23-30). For example, the ∼1.17 Mb heterochromatic knob inversion on chromosome 4 prevents crossover in both wildtype and cmt3 populations (Fransz PF, et al. Cell 2000; 100: 367-76) (FIG. 1D). The chromosome arms showed a significant decrease in crossovers in cmt3 (X² P=2.61×10⁻⁶), which is consistent with a compensatory homeostatic adjustment (FIGS. 1D, 1E, 1G). The location of crossover events was analyzed in relation to gene, levels of CHG DNA methylation and H3K9me2 (Stroud H, et al. Cell 2013; 152: 352-64; and Inagaki S, et al. EMBO J. 2010; 29: 3496-506). Pericentromeric crossovers in both wildtype and cmt3 occurred in regions with lower DNA methylation and H3K9me2 than expected at random.

The following Examples described how to modify H3K9me2 and non-CG DNA methylation during meiosis using artificial microRNA transgenes in maize F₁ hybrids, or maize lines with mixed germplasm, in order to elicit recombination in ordinarily recombination-suppressed regions.

### Example 2. Artificial MicroRNA (amiRNA) Targeting Components of the H3K9me2 and Non-CG DNA Methylation Pathway

Maize components of the H3K9me2 and non-CG DNA methylation pathway include, but are not limited to, the chromomethylase genes ZMET2 (GRMZM2G025592), ZMET5 (GRMZM2G005310) and the maize homologue of KRYPTONITE (GRMZM2G336909). Artificial microRNAs were designed against these genes from the sequences in the reference maize genome (B73 background) using Web MicroRNA designer 3. The Zea mays genome version used was Zea Mays ZmB73 v5b (MGC). See SEQ ID NO: 1-30, below.

### Example 3. Constructs That Specifically Express amiRNA in Meiotic Tissues

amiRNAs that specifically target components of the H3K9me2 and non-CG DNA methylation pathway are expressed under a promoter such that the amiRNAs are active during meiosis in order to reduce expression of the target gene during meiosis. This can be accomplished using a constitutively active promoter or a promoter specifically active (primarily only active) during meiosis. Down regulation of these factors specifically in meiosis, in some embodiments, is preferred, because down regulation during other stages of plant development may have undesirable phenotypic effects (Li Q, et al. The Plant Cell, 2015; 26(12): 4602-16). The amiRNA may be expressed under maize promoters that are specifically active in meiosis, including, but not limited to, AMEIOTIC1 (GRMZM5G883855; Pawlowski WP, et al. PNAS 2008; 106(9): 3603-08), ABSENCE OF FIRST DIVISION (AFD1/REC8) (GRMZM2G059037; Golubovskaya IN, et al. Journal of Cell Science 2006; 119: 3306-3315), and DMC1 (GRMZM2G109618; Etedali F, International journal of Agronomy and Plant Production 2012; 3(3): 100-108), and PHS1 (GRMZM2G100103; Ronceret A, et al. PNAS 2009; 106(47): 20121-26).

The amiRNA may be expressed, for example, as part of a backbone based on the maize microRNA gene zma-miR396h (Meng X, et al. The Plant Cell 2011; 23(3): 942-60). The amiRNA sequence may be inserted into the zma-miR396h gene in place of the endogenous miR396 sequence. As another example, the amiRNA may be expressed as part of a backbone based on a rice microRNA gene Oryza sativa MIR528 (osa-miR528) that has been modified for amiRNA production (Yan F, et al. J of Biotech. 2012; 160(3-4): 146-50).

### Example 4. Transform AmiRNA Expression Cassette Into Maize to Downregulate H3K9me2 and Non-CG DNA Methylation in Maize Meiosis

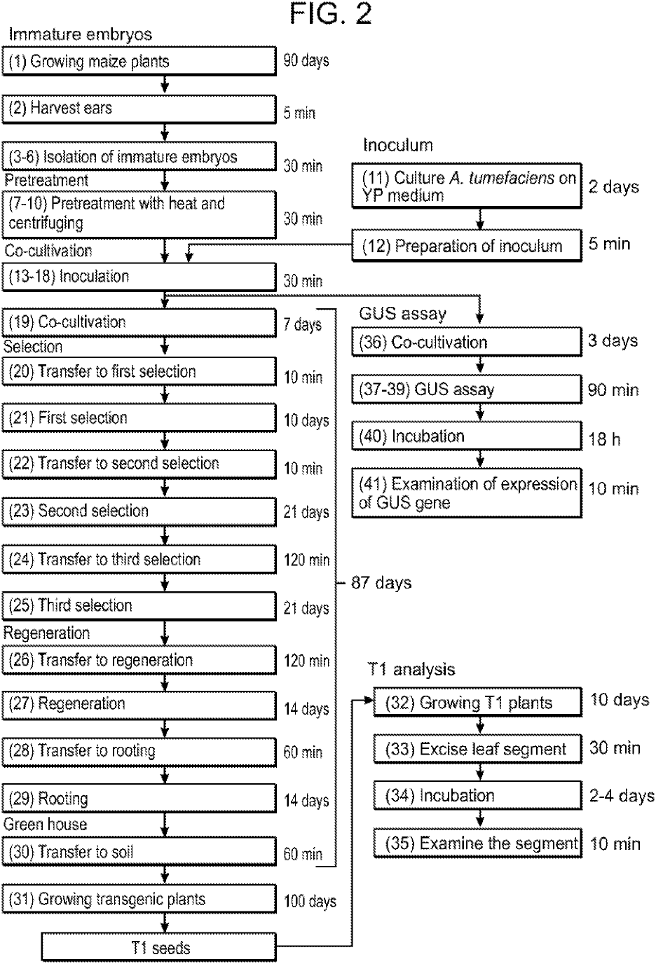

Constructs that express an amiRNA targeting a component of the H3K9me2 and non-CG DNA methylation pathway under a meiotic promoter are transformed into maize using standard approaches (e.g., by agrobacterium mediated transformation of maize embryos followed by plant regeneration (Ishida Y et al. Nature Protocols 2007: 2: 1614-21), by particle bombardment or similar approaches). FIG. 2 shows a flow diagram of a timeline of a maize transformation protocol (Ishida Y et al. Nature Protocols 2007: 2: 1614-21).

The transformation may be carried out on maize inbreds, F₁ hybrids or maize lines with mixed germplasm (e.g., F₂ generation or a later generation). In some embodiments, inbred lines (e.g., B73) are preferred.

A F₁ hybrid plant, or plant with mixed germplasm, where an amiRNA targets a component of the H3K9me2 and non-CG DNA methylation pathway is the key intermediate where novel recombination events (in ordinarily recombination suppressed regions) may occur during meiosis. The seeds that are produced from this plant are the final product of sexual reproduction and they can be used to screen for novel recombination events, which are unlikely to occur in wild type control crosses.

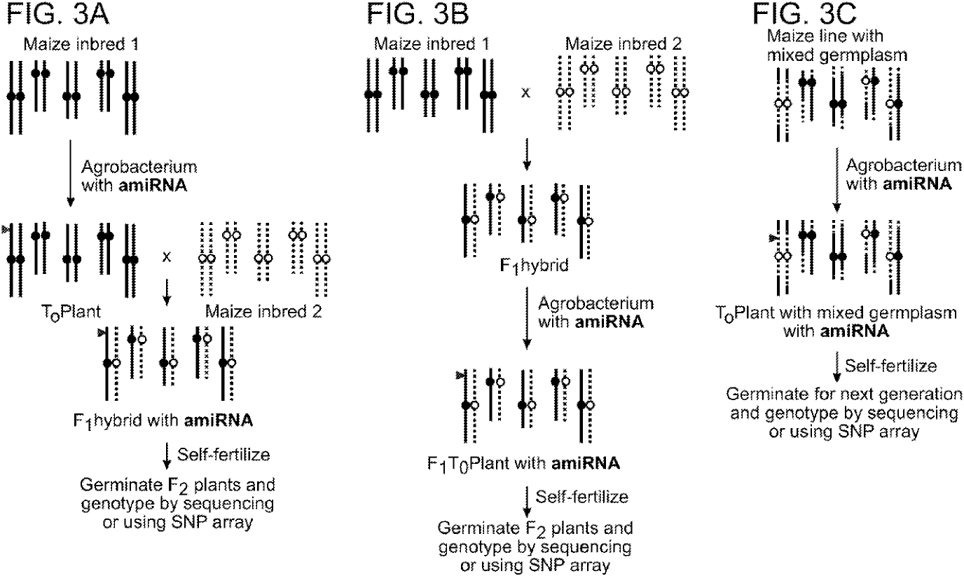

### Protocol for Using an Inbred for Transformation (FIG. 3A):

a) An amiRNA targeting H3K9me2 and non-CG DNA methylation pathway component is transformed into inbred maize variety (e.g., B73) and a T₀ plant is regenerated.

b) A T₀ plant, or derivative, is crossed to a divergent maize variety (e.g., Missouri 17).

c) The product of the cross, the F₁ seed, is harvested and then planted and grown to maturation.

d) A F₁ hybrid plant is self-pollinated to produce F₂ seed (or is backcrossed to a parental line or outcrossed to a non-parental inbred line).

e) F₂ seeds (or equivalent seeds derived from a backcross or outcross) are used to develop a large mapping population (e.g., of 100 individuals or more) by germinating seed, growing plants and extracting DNA.

f) Novel recombination events, in ordinarily recombination suppressed regions, are screened. Genotyping of a mapping population may be carried out using, for example, a maize SNP array (Ganal MW, et al. PLOS One 2011; 6(12): e28334) or by ILLUMINA®-based genotyping by sequencing.

### Protocol for Using a Hybrid for Transformation (FIG. 3B)

a) An amiRNA targeting H3K9me2 and non-CG DNA methylation pathway component is transformed directly into F₁ hybrid maize (e.g., B73 x Missouri 17 F₁) and a T₀ plant is regenerated.

b) The F₁T₀ plant is grown to maturation.

c) A F₁T₀ plant is self-pollinated to produce F₂ seed (or is backcrossed to a parental line or outcrossed to a non-parental inbred line).

d) F₂ seeds (or equivalent seeds derived from outcross) are used to develop a large mapping population (e.g., of 100 individuals or more) by germinating seed, growing plants and extracting DNA.

e) Novel recombination events, in ordinarily recombination suppressed regions, are screened. Genotyping of a mapping population may be carried out using, for example, a maize SNP array (Ganal MW, et al. PLOS One 2011; 6(12): e28334) or by ILLUMINA®-based genotyping by sequencing.

### Protocol for Using a Later Generation (e.g., F₂ or Later) for Transformation (FIG. 3C)

a) An amiRNA targeting a H3K9me2 and non-CG DNA methylation pathway component is transformed into a plant with mixed germplasm (e.g., a B73 x Missouri 17 F₂ line - a second generation post crossing) and a T₀ plant regenerated.

b) A T₀ plant is grown to maturation.

c) A T₀ plant is be self-pollinated to produce seed (or is backcrossed to a parental line or outcrossed to a non-parental inbred line).

d) Seeds from self- pollination (or equivalent seeds derived from outcross) are used to develop a large mapping population (e.g., of 100 individuals or more) by germinating seed, growing plants and extracting DNA.

e) Novel recombination events, in ordinarily recombination suppressed regions, are screened. Genotyping of a mapping population may be carried out using, for example, a maize SNP array (Ganal MW, et al. PLOS One 2011; 6(12): e28334) or by ILLUMINA®-based genotyping by sequencing.

The following Examples described how to modify H3K9me2 and non-CG DNA methylation during meiosis using virus-induced gene silencing (VIGS) in maize F₁ hybrids, or maize lines with mixed germplasm, in order to elicit recombination in ordinarily recombination-suppressed regions.

### Example 5. Virus-Induced Gene Silencing (VIGS) in Maize to Downregulate H3K9me2 and Non-CG DNA Methylation in Maize Meiosis

Maize components of the H3K9me2 and non-CG DNA methylation pathway include the chromomethylase genes ZMET2 (GRMZM2G025592), ZMET5 (GRMZM2G005310) and the maize homologue of KRYPTONITE (GRMZM2G336909). Sequences for introduction into a VIGS vector were designed against these genes using the cDNA sequences in Gramene’s B73 RefGen_v3 sequence: GRMZM2G025592_T01; GRMZM2G005310_T01; and GRMZM2G336909_T01. These cDNA sequences were inputted into a VIGS prediction software (vigs.solgenomics.net) that minimizes off target effects, by comparing to the reference Maize transcriptome Zea mays B73 v5a. Parameters included: fragment size, 300; n-mer, 21; mismatches, 0; and database, Zea_mays_B73_v5a. See SEQ ID NO: 31-33, below. Also described below is a sequence (SEQ ID NO: 34) derived from ZMET2 (GRMZM2G025592_T01), which when inserted into a VIGS vector also targets ZMET5 (GRMZM2G005310_T01). A VIGS vector using this sequence may lead to concurrent knockdown of both ZMET2 and ZMET5.

### Example 6. VIGS Vectors for Infection of Maize to Initiate Knockdown of the H3K9me2 and Non-CG DNA Methylation Pathway

VIGS vectors are based on the maize-infecting cucumber mosaic virus (CMV) strain, ZMBJ-CMV (Wang R, et al. Plant J. 2016; 86(1):102-15, incorporated by reference). ZMBJ-CMV has been modified to facilitate efficient VIGS in maize (Wang R, et al. Plant J. 2016; 86(1):102-15). This method modifies the CMV RNA2 genome by introducing 100-500 nucleotide fragments of the target gene downstream of the 3′ of the open reading frame (ORF) expressing the 2b protein. Other viruses may be modified in a similar manner to include sequences corresponding to ZMET2 (GRMZM2G025592), ZMET5 (GRMZM2G005310) and KRYPTONITE (GRMZM2G336909).

### Example 7. Infect Maize With Virus Designed to Elicit Knockdown of H3K9me2 and Non-CG DNA Methylation

DNA constructs that contain the modified viral genome are transformed into Agrobacterium tumefaciens (Wang R, et al. Plant J. 2016; 86(1):102-15). This Agrobacterium is used to inoculate maize directly at kernel, seedling, plantlet or other stage. The Agrobacterium inoculation leads to virus production, which elicits VIGS of the target gene.

The Agrobacterium is also used to inoculate N. benthamiana, a plant species that is capable of high viral titers. Crude sap from the inoculated N. benthamiana is then prepared (4 days after agro infiltration), which is used to infect maize at kernel, seedling, plantlet or other stage. This elicits VIGS of the target gene.

Viral infection and VIGS is carried out on maize F₁ hybrids or maize lines with mixed germplasm (e.g., F₂ generation or later).

The F₁ hybrid plant, or plant with mixed germplasm, where a VIGS vector targets a component of the H3K9me2 and non-CG DNA methylation pathway, is the key intermediate where novel recombination events (in ordinarily recombination suppressed regions) occur during meiosis. The seeds that are produced from this plant are the final product of sexual reproduction and they are used to screen for novel recombination events, which are unlikely to occur in wild type control crosses.

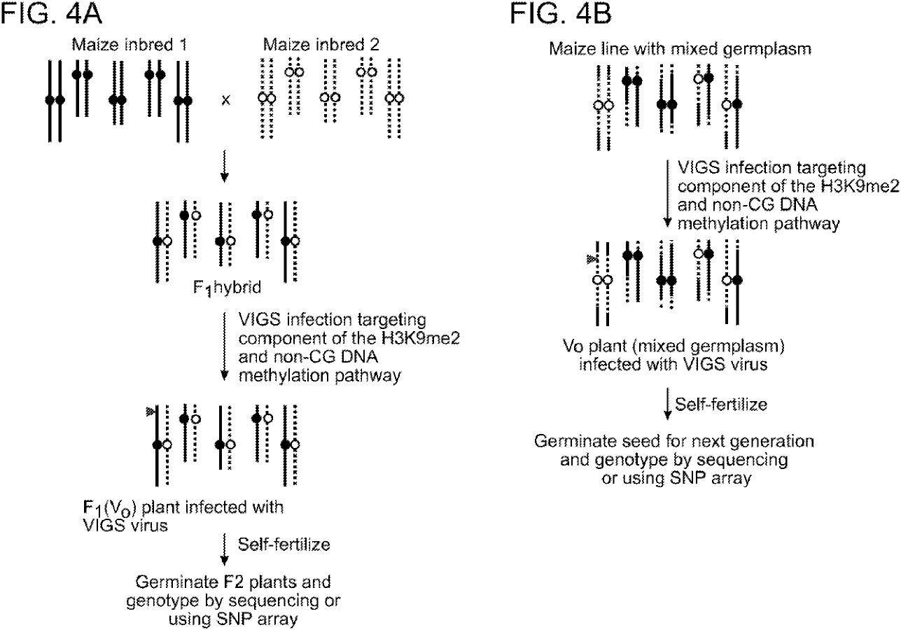

### Protocol for Using a Hybrid for Infection (See FIG. 4A)

a) A VIGS virus targeting H3K9me2 and non-CG DNA methylation pathway component is used to infect F₁ hybrid maize (e.g., B73 x Missouri 17 F₁).

b) The F₁ (V₀) plant is grown to maturation.

c) The F₁ (V₀) plant is self-fertilized to produce F₂ seed (or is outcrossed to a parental or non-parental inbred line).

d) The F₂ seeds (or equivalent seeds derived from outcross) are used to develop a large mapping population (e.g., of 100 individuals or more) by germinating seed, growing plants up and extracting DNA.

f) Novel recombination events, in ordinarily recombination suppressed regions, are screened. Genotyping of a mapping population may be carried out using, for example, a maize SNP array (Ganal MW, et al. PLOS One 2011; 6(12): e28334) or by ILLUMINA®-based genotyping by sequencing.

### Protocol for Using a Plant With Mixed Germplasm (e.g., F₂ Generation or Later) for Infection) (See FIG. 4B)

a) A VIGS virus targeting H3K9me2 and non-CG DNA methylation pathway component is used to infect plant with mixed germplasm (e.g., a B73 x Missouri 17 F₂ line - i.e. second generation post crossing).

b) The V₀ plant is grown to maturation.

c) The V₀ plant is self-fertilized to produce seed (or is to a parental or non-parental inbred line).

d) Seeds from self-fertilization (or equivalent seeds derived from outcross) are used to develop a large mapping population (e.g., of 100 individuals or more) by germinating seed, growing plants up and extracting DNA.

e) Novel recombination events, in ordinarily recombination suppressed regions, are screened. Genotyping of a mapping population may be carried out using, for example, a maize SNP array (Ganal MW, et al. PLOS One 2011; 6(12): e28334) or by ILLUMINA®-based genotyping by sequencing.

### Example 8. Detecting Meiotic Crossovers by Examining Chiasmata in Maize Meiotic Cells

Chiasmata are the physical manifestations of meiotic crossovers and can be detected by mounting and spreading meiotic cells in the presence of a DNA stain (e.g., DAPI) followed by visualization of the chromosomes by fluorescence microscopy. In maize, most chiasmata are located at the tips (“subtelomeric regions”) of the chromosomes, where gene density is highest (Anderson LK et al. Genetics. 2003; 165: 849-65; Sidhu GK, et al. PNAS 2015; 112: 15982-87; and Rodgers-Melnick E, et al. PNAS USA 2015: 112: 3823-28). In contrast, the central parts of the chromosomes tend not to recombine, due to their highly repetitive nature. Therefore, chiasmata are generally not found in central parts of maize chromosomes in what is known as “pericenteromeric heterochromatin” (Sidhu GK, et al. PNAS 2015; 112: 15982-87; and Rodgers-Melnick E, et al. PNAS USA 2015: 112: 3823-28).

A detailed protocol that makes it possible to examine chiasmata in maize has recently been described (Sidhu GK, et al. PNAS 2015; 112: 15982-87).

In order to assay whether knockdown of H3K9me2 or non-CG DNA methylation factors has an effect on crossover distribution directly in an F₁ hybrid plant, the spatial distribution of chiasmata along the lengths of chromosomes is examined in wild type and knock down meiotic cells (Sidhu GK, et al. PNAS 2015; 112: 15982-87). It is possible to determine chiasmata location along the length of a chromosome by visual inspection of meiotic bivalents.

In wild type plants, almost all chiasmata are present at the tips of chromosomes (“subtelomeric regions”), and not in central chromosomal regions (“pericentromeric heterochromatin”) (Sidhu GK, et al. PNAS 2015; 112: 15982-87).

In the H3K9me2/non-CG DNA methylation knockdown lines, chiasmata may be detected in central chromosomal regions (“pericentromeric heterochromatin”).

For further validation of location of chiasmata relative to maize centromeres, chiasmata detection may be combined with Fluorescence In Situ Hybridisation (FISH) to locate the position of centromeres on the chromosomes. By using fluorescently labeled oligonucleotide probes against the maize centromeric repeats, CentC and CRM (Jin W, et al. PLANT CELL ONLINE 2004; 16: 571-81), maize centromeres can be specifically labeled. Proximity of chiasmata location in wild type and H3K9me2/non-CG DNA methylation knockdown lines to centromeres shows whether the desired increase in pericenteromeric recombination has occurred in the H3K9me2/non-CG DNA methylation knockdown lines.

### Example 9. Quantification of Knockdown Efficiency of H3K9me2/Non-CG DNA Methylation Factor Gene Expression in Maize Anthers

To have an effect on meiotic recombination, H3K9me2/non-CG DNA methylation factor gene expression should be reduced in meiotic cells. Maize anthers, the male sexual organs, can contain a high proportion of meiotic cells, when the correct stage of anther development is analyzed. Reduction in gene expression of a H3K9me2/non-CG DNA methylation factor, in maize anthers that harbor a gene-silencing cassette, may be assessed using the following approach:
1) Extract RNA from developmentally staged maize anthers;
2) Produce complementary DNA (cDNA) based on the RNA, using a reverse transcriptase reaction; and
3) Quantify gene expression using quantitative reverse transcript polymerase chain reaction (qRT-PCR)

### RNA Extraction From Maize Anthers

Maize anthers between 1000-1200 µm are dissected and collected, as these anthers are most enriched for pollen mother cells (meiotic cells) (Kelliher T and Walbot V, Dev. Biol. 2011; 350: 32-49). Collected tissue is frozen in liquid nitrogen and ground to a fine powder using a pre-frozen pestle and mortar. RNA extraction is carried out using Plant RNA reagent (Invitrogen), according to manufacturer’s protocol, including a DNase treatment step. RNA concentration is quantified (e.g., by using a Qubit® fluorometer (ThermoFisher), in accordance with the manufacturers protocol).

### Complementary DNA (CDNA) Preparation

Equal amounts of RNA are used to prepare cDNA by incubating RNA in the presence of a reverse transcriptase enzyme (e.g., SuperScript® (ThermoFisher), in accordance with the manufacturers protocol). The reaction is primed with either random hexamer primers or oligo(dT) primers. Control reactions are carried out without reverse transcriptase present. Gene expression quantification by quantitative reverse transcript PCR (qRT-PCR)

qRT-PCR is used to quantify gene expression of the target gene. Primers that specifically amplify the target gene and a housekeeping gene are. cDNA is PCR amplified in separate reactions containing either the target gene specific primers or housekeeping gene specific primers. Amplification is carried out in a quantitative PCR (qPCR) machine, in the presence of a fluorescent DNA stain (e.g., SYBR® Green reagent (ThermoFisher), according to manufacturer’s protocol). ΔΔCt values are calculated and used to determine changes of H3K9me2/non-CG DNA methylation factor gene expression in knockdown and control samples.

### SEQUENCES

### ZMET2 (GRMZM2G025592_T01)

TTTCTGTACCGATTAAACCCG (SEQ ID NO: 1)

TTTCTGTACCGATAAAACCCG (SEQ ID NO: 2)

TTAAATGTTACCTGGTGACTG (SEQ ID NO: 3)

TTTTAGATTAGATACCGTCAC (SEQ ID NO: 4)

TTTTTTAACGGACTCGGTCAA (SEQ ID NO: 5)

TCATAGTATAGGTTGCGACTC (SEQ ID NO: 6)

TTAGTATAGGTCGTACCTCTC (SEQ ID NO: 7)

TTAACATATCCCGAGAAGCTT (SEQ ID NO: 8)

TTAAATGTTACCTGATGACTC (SEQ ID NO: 9)

TACGAAGCATCAATACGGCTG (SEQ ID NO: 10)

### ZMET5 (GRMZM2G005310_T01)

TATGTTAGTAACACGTGGCCG (SEQ ID NO: 11)

TTCAAGAGCTGTAATTGACTT (SEQ ID NO: 12)

TATGTTAGTAACACATGGCCG (SEQ ID NO: 13)

TTCAAGAGCTGTAATTGGCTT (SEQ ID NO: 14)

TCTATGGGTCTAAAGTACCTA (SEQ ID NO: 15)

TCTATGGGTCTAAAGCGCCTA (SEQ ID NO: 16)

TTTAACAGCTGACAATTGCTA (SEQ ID NO: 17)

TTTAACAGCTGACAACCGCTA (SEQ ID NO: 18)

TTTAACAGCTGACAACTCCGA (SEQ ID NO: 19)

TTGTTAGTAACACAGCGCCGT (SEQ ID NO: 20)

### Kryptonite (GRMZM2G336909_t01)

TTAATCGAAGATTGTCCGCAA (SEQ ID NO: 21)

TTAATCGAAGATTGTTGGCAA (SEQ ID NO: 22)

TATTGTCGGCAAAATATGCTG (SEQ ID NO: 23)

TAGTTAATCGAAGACTGTCGG (SEQ ID NO: 24)

TAGTTAATCGAAGATTCTCGG (SEQ ID NO: 25)

TATTGTCGGCAAAATACGCTA (SEQ ID NO: 26)

TTACAATATAAGCTGTCTCTT (SEQ ID NO: 27)

TTACAATATAAGCTCTTTCCT (SEQ ID NO: 28)

TTAGAGTGAGCGTCTATCCAA (SEQ ID NO: 29)

TATTGTCGGCAAAATATGCTA (SEQ ID NO: 30)

### GRMZM2G025592_T01_(1297-1596)

CGATGAGTTTCTTGCCCTCCTTAAGGAATGGGCAGTTCTATGCAAAAAAT
ATGTCCAAGATGTGGATTCAAATTTAGCAAGCTCAGAGGATCAAGCGGAT
GAAGACAGCCCTCTTGACAAGGACGAATTTGTTGTAGAGAAGCTTGTCGG
GATATGTTATGGTGGCAGTGACAGGGAAAATGGCATCTATTTTAAGGTCC
AGTGGGAAGGATACGGCCCTGAGGAGGATACATGGGAACCGATTGATAAC
TTGAGTGACTGCCCGCAGAAAATTAGAGATTTTGTACAAGAAGGGCACAA
(SEQ IDNO: 31)

### GRMZM2G005310_T01_(1234-1533)

TGATGAGTTTCTTGCTCTCCTTAAGGAATGGGCAGTCTTATGTGAAAAAT
ATGTTCATCAAGACGTGGATTCGAATTTAGCAGGTTCAGAGGATCAAGAG
GATGCTGACACTCTTGACAAGGATGAATTTGTTGTACAGAAGCTTATTGG
AATACGCTATGATGGCACTGGAAGGAAAAAAGGCGTATATTTTAAGGTCC
AATGGGAAGGATATGGTCCTGAAGAGGATACATGGGAACCTATTGATAAC
CTGAGTGACTGTCCGCTGAAAATTAGAGAATTTGTACAAGAAGGGCGCAA
(SEQ IDNO: 32)

### GRMZM2G336909_T01_(1947-2246)

AAAAATTGTGAAGCTGGCCTGCCACTGTGGTGCTCCTGATTGCCGGAAGC
GCCTCTACTAACCTGCAGTGTGTTTATTTCTATTTGGATTTTTGAGGAAA
CAGCTTATATTGTAGGAAAAACAGTCATGGTACCTATGTAGTTGACATGC
TCTTGATCATATATAGCAAAAGAGGAGCAATTTATGGGGTGTTCGGTTTA
AGAATGAAATTAGAGATTTAGAGGACATGGTGGTCATGGTAGCATATTTT
GCCGACAATCTTCGATTAACTGATATATCTGGACATCTGGTCCTGCAACA
(SEQ IDNO: 33)

### GRMZM2G025592_T01_(960-1296)

GATAGAGAGTTGCGACCTATACTATGACATGTCTTACTCTGTTGCATATT
CTACATTTGCTAATATCTCGTCTGAAAATGGGCAGTCAGACAGTGATACC
GCTTCGGGTATTTCTTCTGATGATGTGGATCTGGAGACGTCATCTAGTAT
GCCAACGAGGACAGCAACCCTTCTTGATCTGTATTCTGGCTGTGGGGGCA
TGTCTACTGGTCTTTGCTTGGGTGCAGCTCTTTCTGGCTTGAAACTTGAA
ACTCGATGGGCTGTTGATTTCAACAGTTTTGCGTGCCAAAGTTTAAAATA
TAATCATCCACAGACTGAGGTGCGAAATGAGAAAGC (SEQ ID NO: 3
4)

### GRMZM2G336909_T01 (Kryptonite)

ATGCCGTACGAAAGCTGGTGGACGCCGAAGGTGAATTCGGTGGCGCCGGT
GCCGAACCCGATATTCATGGACGCCTCTCCCCTGAACCCGCCGCCGCTGG
TGGAGGAGGAGCTTGTGCTGCGCCGCAGTGCTCGTTGCCTCAATAGGCCA
ACCCGGCCCAGCTACGCGGAACAGGAGCCTCCTAAAAAACCCGGCGGCCG
GGGCCGGAGCAAGCGGAAGAGGGACGAGGAGAAACCGGAGCTGGCACCGG
CGCAGCAGGGTGCCAAGAGTCCGGACAGGAAGGACTCCAAGGATGAGGCG
GGTGAGAAGAAGCCGACGCCCATGATTGCGGCGCTGCCGGTCTCTTGCGC
TGGCGTTGCTGCTGAAGACGATGCGACGGGGACCGGGAAGAGCGCCAAGC
TGAGGGTGAAGGAGACACTGAGGGCGTTCAATAGCCACTACCTCCACTTT
GTGCAGGAGGAGCAGAAGAGAGCTCAGGCTGCGCTACAGGAGAAACGCCC
ATCAAAGCGGCCTGACCTGAAAGCAATCACCAAGATGCAAGAAATGAATG
CCGTGCTTTATCCAGAGAAAACGATTGGACATCTACCAGGGATCGATGTT
GGAGATCATTTCTATTCTCGCGCTGAAATGGTAGTGTTGGGCATACACAG
CCATTGGCTCAATGGCATTGACTTCATGGGGCTGAAATACCAAGGAAAGG
AGTATTCAAACTTAACTTTCCCACTGGCCACCTGTATCGTAATGTCGGGG
GTATATGAAGATGATCTAGACAAAGCTGATGAAATTATTTATACTGGTCA
AGGAGGAAATGATTTGCTTGGTAACCATCGCCAAATTGGCTCTCAACAGT
TGAAACGTGGGAATTTAGCGTTGAAGAATAGCAGGGAGAATGGTAATCCT
GTCCGAGTTGTTCGAGGACATTTGTCAAAGAATAGCTACACTGGAAAGAT
CTACACCTATGATGGGCTTTACAAGGTTGTAGATGACTGGGTGCAGAAGG
GAGTGCAAGGCCATGTTGTTTTCAAATTTAAGTTGAAGCGGCTTGAGGGT
CAGCCATCATTGACAACCTCTGAGGTGCGATTTACTCGTGCAGAAGCTCC
GACAACTATTTCCGAATTACCCGGGCTGGTTTGTGATGACATCTCTGGAG
GGCAAGAAAATATTCCTATTCCTGCTACTAACTTGGTTGATGATCCACCT
GTTCCTCCATCTGGTTTCAAGTACTTGAAATCTCTACAAATTCCAAAGGA
CATCAAGATTCCGTCTAGTATTATTGGCTGTGATTGTGAAGGAGGTTGTG
CTAGCAACAAAAAATGTTTGTGTGCTCAGCGCAATGGTTCTGATCTGCCT
TATGTATCATATAAGAACATTGGCAGGTTGGTGGAGCCGAAAGCAGTTGT
ATTTGAATGTGGGGCTAACTGCAGCTGCAACCATGATTGTGTAAACAGAA
CATCTCAACAAGGTTTGCAGTACCGCTTAGAGGTATTTAAGACAGCTTCA
AAAGGATGGGGAGTCAGGACTTGGGACACTATCCTCCCTGGGGCTCCCAT
CTGTGAGTACACAGGGGTGTTGAGGAGGACCGAAGATTTGGATGGTTCAC
AGAACAACTATTGTTTTGACATTGACTGTCTTCAAACCATGAAGGGTTTG
GATGGAAGGGAGAAAAGGGCTGGATCCGAAATGCACCTGCCTAATCTTCA
TCCTGAGAATGATTCAGATGCACAACCTGCACCTGAGTACTGCATAGACG
CTCACTCTATTGGCAACTTTGCAAGATTTATAAATCACAGCTGCCAACCT
AACCTTTTCGTCCAGTGCGTCTTGAGCTCACACAATGATGTTAAACTGGC
GAAAGTGATGCTTTTCGCTGCTGACACCATACTTCCTCTTCAGGAGCTAT
CCTATGACTACGGTTATCGATTGGACAGTGTTGTTGGGCCTGATGGAAAA
ATTGTGAAGCTGGCCTGCCACTGTGGTGCTCCTGATTGCCGGAAGCGCCT
CTACTAACCTGCAGTGTGTTTATTTCTATTTGGATTTTTGAGGAAACAGC
TTATATTGTAGGAAAAACAGTCATGGTACCTATGTAGTTGACATGCTCTT
GATCATATATAGCAAAAGAGGAGCAATTTATGGGGTGTTCGGTTTAAGAA
TGAAATTAGAGATTTAGAGGACATGGTGGTCATGGTAGCATATTTTGCCG
ACAATCTTCGATTAACTGATATATCTGGACATCTGGTCCTGCAACACTTA
TCCTCTGGATAAATATATATACGTTACA (SEQ ID NO: 35)

### GRMZM2G025592_T01 (zmet2)

ATTCGATTTCTACACCTCCAGAGCACTCACTCTCCTGGCAAACCCCTTCG
TCTCCCAACCCTAGAGAGCAGCAGCAGCTACCGCAGCCCCTGCCATGGCG
CCGAGCTCCCCGTCACCCGCCGCGCCCACACGCGTCTCTGGGCGGAAGCG
CGCCGCCAAGGCCGAGGAGATCCACCAGAACAAGGAGGAGGAGGAGGAGG
AGGAGGAGGTCGTGGCGGCGTCCTCCGCCAAGCGCAGCCGCAAGGCGGCA
TCTTCTGGGAAGAAGCCCAAGTCGCCCCCCAAGCAGGCCAAGCCGGGGAG
GAAGAAGAAGGGGGATGCCGAGATGAAGGAGCCCGTGGAGGACGACGTGT
GCGCCGAGGAGCCCGACGAGGAGGAGTTGGCCATGGGCGAGGAGGAGGCC
GAGGAGCAGGCCATGCGGGAGGAGGTGGTTGCGGTCGCGGCGGGGTCACC
CGGGAAGAAGAGGGTGGGGAGAAGGAACGCCGCCGCCGCCGCCGGCGACC
ACGAGCCGGAGTTCATCGGCAGCCCTGTTGCCGCGGACGAGGCGCGCAGC
AACTGGCCCAAGCGCTACGGCCGCAGCACTGCCGCAAAGAAACCGGATGA
GGAGGAAGAGCTCAAGGCCAGATGTCACTACCGGAGCGCTAAGGTGGACA
ACGTCGTCTACTGCCTTGGGGATGACGTCTATGTCAAGGCTGGAGAAAAC
GAGGCAGATTACATTGGCCGCATTACTGAATTTTTTGAGGGGACTGACCA
GTGTCACTATTTTACTTGCCGTTGGTTCTTCCGAGCAGAGGACACGGTTA
TCAATTCTTTGGTGTCCATAAGTGTGGATGGCCACAAGCATGACCCTAGA
CGTGTTTTTCTTTCTGAGGAAAAGAACGACAATGTGCTTGATTGCATTAT
CTCCAAGGTCAAGATAGTCCATGTTGATCCAAATATGGATCCAAAAGCCA
AGGCTCAGCTGATAGAGAGTTGCGACCTATACTATGACATGTCTTACTCT
GTTGCATATTCTACATTTGCTAATATCTCGTCTGAAAATGGGCAGTCAGA
CAGTGATACCGCTTCGGGTATTTCTTCTGATGATGTGGATCTGGAGACGT
CATCTAGTATGCCAACGAGGACAGCAACCCTTCTTGATCTGTATTCTGGC
TGTGGGGGCATGTCTACTGGTCTTTGCTTGGGTGCAGCTCTTTCTGGCTT
GAAACTTGAAACTCGATGGGCTGTTGATTTCAACAGTTTTGCGTGCCAAA
GTTTAAAATATAATCATCCACAGACTGAGGTGCGAAATGAGAAAGCCGAT
GAGTTTCTTGCCCTCCTTAAGGAATGGGCAGTTCTATGCAAAAAATATGT
CCAAGATGTGGATTCAAATTTAGCAAGCTCAGAGGATCAAGCGGATGAAG
ACAGCCCTCTTGACAAGGACGAATTTGTTGTAGAGAAGCTTGTCGGGATA
TGTTATGGTGGCAGTGACAGGGAAAATGGCATCTATTTTAAGGTCCAGTG
GGAAGGATACGGCCCTGAGGAGGATACATGGGAACCGATTGATAACTTGA
GTGACTGCCCGCAGAAAATTAGAGATTTTGTACAAGAAGGGCACAAAAGA
AAGATTCTCCCACTGCCTGGTGATGTTGATGTCATTTGTGGAGGCCCACC
ATGCCAAGGTATCAGTGGGTTTAATCGGTACAGAAACCGTGATGAGCCAC
TCAAAGATGAGAAAAACAAACAAATGGTGACTTTCATGGATATTGTGGCG
TACTTGAAGCCCAAGTATGTTCTCATGGAAAATGTGGTGGACATACTCAA
ATTTGCGGATGGTTACCTAGGAAAATATGCTTTGAGCTGCCTTGTTGCTA
TGAAGTACCAAGCGCGGCTTGGAATGATGGTGGCTGGTTGCTATGGTCTG
CCACAGTTCAGGATGCGTGTGTTCCTCTGGGGTGCTCTTTCTTCCATGGT
GCTCCCTAAGTATCCTCTGCCCACCTATGATGTTGTAGTACGTGGAGGAG
CCCCTAATGCCTTTTCGCAATGTATGGTTGCATATGACGAGACACAAAAA
CCATCCCTGAAAAAAGCCTTGCTTCTTGGCGATGCAATTTCAGATTTACC
AAAGGTTCAAAATCACCAGCCTAACGATGTGATGGAGTATGGTGGTTCCC
CCAAGACCGAATTCCAGCGCTACATTCGACTCAGTCGTAAAGACATGTTG
GATTGGTCCTTCGGTGAGGGGGCTGGTCCAGATGAAGGCAAGCTCTTGGA
TCACCAGCCTTTACGGCTTAACAACGATGATTATGAGCGGGTTCAACAGA
TTCCTGTCAAGAAGGGAGCCAACTTCCGCGACCTAAAGGGCGTGAGGGTT
GGAGCAAACAATATTGTTGAGTGGGATCCAGAAATCGAGCGTGTGAAACT
TTCATCTGGGAAACCACTGGTTCCTGACTATGCAATGTCATTCATCAAGG
GCAAATCACTCAAGCCGTTTGGGCGCCTGTGGTGGGACGAGACAGTTCCT
ACAGTTGTAACCAGAGCAGAGCCTCACAACCAGGTTATAATTCATCCGAC
TCAAGCAAGGGTCCTCACTATCCGGGAGAACGCAAGGTTACAGGGCTTCC
CCGATTACTACCGATTGTTTGGCCCGATCAAGGAGAAGTACATTCAAGTC
GGGAACGCAGTGGCTGTCCCTGTTGCCCGGGCACTGGGCTACTGTCTGGG
GCAAGCCTACCTGGGTGAATCTGAGGGGAGTGACCCTCTGTACCAGCTGC
CTCCAAGTTTCACCTCTGTTGGAGGACGCACTGCGGGGCAGGCGAGGGCC
TCTCCTGTTGGCACCCCTGCAGGGGAGGTAGTTGAGCAGTAAAAGGATGA
CAGATCTGAGCTGAGCTGGGCAACATCCAGCGGCAGGAGCATTTCTGGTT
CGGTTCGATTCGGGCTCACGAGCGCATTGTGCTGATTCGCAGGGTGCACT
CGAGCTCGAGTGCGGAGTTGTTGTAATGTAGGTTGAGGTTTCAAAACATC
CATCCATCCATCGGCCCCGCCTATTTGTGGATGGTGTTTCTCGTGCCATT
TTAGTTCAGCTGTATTGATGCTTCGTCATGTATGTGTATTGTACCATTCG
TTCTTGTTACCTACTAGTAAGGCAACATTTGATCCATGTTCAACGCGAGC
AAAATTCCATCCACTTTAGGTACTGAGATGCTCAAGTAGTTCAGTCATCA
GGTAACATTTACAACCTATTATTTTGCCTGGTATTATCTGAAAACGATTT
GCTGAGGATGTTTTTTTTTTGAAGTAAAGGTACTGTAGGAAGGTCCTACA
GTAATATTATTAAATCAAAAGAACTAAGTTTAGGGAGACAGAAGAATATT
ACAAAGAAATAAAAGGGAGAGTGAGGAGGGGGAAGAATTCTGGATACATG
TATTTACTCTATTTCACATAAACATATGGACATCAACATAGATGTGAAGG
TATCTAATCTAAATAAGCGCCGACATGCTTTACAAGATAGTTTGATCTAT
AAGGTGAGGGATGTTGTGGATGAAGATTGTGACCAACTAGCTCATGTGTA
GTTTTGTTTGTAGTACCTGTCCTAAAAAATATGTAATTCTAAGTTTAATT
TGAATCAGCCTTGTTTTTTAAAATTTTTTTGACCAAGTCCGTTAAAAAGT
AATAACATTTTTTACTTCAAATAGATTTACTATGAAATTTTGTCTTATGG
TTATCTAATGACATTTATTTAAAACTGTAACAAAAACTGGTTTGGTTAGG
GGACTGAGGGAGTAGTGCTTAAAATGGAAGACGATAGGAGAGATCACATT
AGTGGTGGC (SEQ ID NO: 36)

### GRMZM2G005310_t01 (Zmet5)

AGCAGCAGCAGCTACCGCATCCCTTGCCATGGCGCCAAGCTCCCCGTCAT
CCGCTAGGCCCACGCGCGCCTCCGGCCGGAAGCGCTCCGCCATGGCCGAG
GAGATCCACCAGAACCAGGAGGAGGAGGAGGAGGTTGTGGCTGCGTCCAC
CGCCAAGCGCAGACGCAAGGCGGCGTCTTCCGGGAAGAAGCCCAAGCCGA
CGCCCAAGCAGGCCAAACCGGCCGTGGCGGGGATGAAGAAGAAGGGGGAG
ACCGAGAAGACGGAGCCCGTTGTGGACGACGTGTGCGCCGAGGAGCCCGA
CGAGGAGGAGTTGGCCATGGGCGAGGAGGAGGCGGAGGCCGAGGAGCAGG
CCATGCAGGAGGTGGTGGCCGCGGTCGCGGCCGGGTCGCCCGGTAAGAAG
AGGGTGGGGAGAAGGAGCGCCGCCGCCTCGGGCGACCACGTGCCCGAGTT
CATCGGCAGCCCCGTTGCTGCGGCCGAGGCGCACAGCAACTGGCCCAAGC
GCTACGAGCGCAGCACTGCCGCAAATAAACCGGAGGAGGACGATGAGCTC
AAGGCCAGGTGCCACTACCGGAGCGCTAAGGTGGACAACATTGTCTACTG
CCTCGGGGATGACGTTTATGTCAAGGCTGGAGAAAATGAGGCTGATTACA
TTGGTCGCATTACTGAATTTTTTGAGGGCACTGATCGATGCCACTATTTT
ACTTGCCGTTGGTTTTTCCGTGCAGAGGACACGGTCATCAATTCGTTGGT
GTCGATTAATGTTGATGGCCACAAGCATGACCCTAGACGTGTTTTTCTTT
CCGAGGAAAAGAATGACAATGTGCTTGATTGCATCATCTCCAAGGTCAAG
ATAGTCCATGTTGATCCAAATATGGATCCAAAAGCTAAGGCCCAACTGAT
AGAACATTGTGACCTATACTATGACATGTCTTACTCTGTTGCATATTCTA
CATTTGCTAATATCTCATCAGAAAATGGGCAGTCAGGCAGTGAAACCGCT
TCGGGTATTTCTTCTGATGATGCGGGTTTGGAGACGTCATCTAATATGCC
AGAGAGGACAGCAACCCTTCTTGACTTGTATTCGGGCTGTGGGGGCATGT
CCACTGGTCTTTGCTTGGGTGCTGCTCTTTCTGGCCTGAAACTTGAAACT
CGATGGGCTGTTGATCTTAACAGTTTTGCGTGCCAAAGTTTAAAATACAA
TCATCCACAGACTGAGGTGCGAAATGAGAAAGCTGATGAGTTTCTTGCTC
TCCTTAAGGAATGGGCAGTCTTATGTGAAAAATATGTTCATCAAGACGTG
GATTCGAATTTAGCAGGTTCAGAGGATCAAGAGGATGCTGACACTCTTGA
CAAGGATGAATTTGTTGTACAGAAGCTTATTGGAATACGCTATGATGGCA
CTGGAAGGAAAAAAGGCGTATATTTTAAGGTCCAATGGGAAGGATATGGT
CCTGAAGAGGATACATGGGAACCTATTGATAACCTGAGTGACTGTCCGCT
GAAAATTAGAGAATTTGTACAAGAAGGGCGCAAAAGAAAAATTCTCCCGC
TTCCTGGTGATGTTGATGTCATTTGTGGAGGCCCACCATGTCAAGGTATA
AGTGGGTTTAATCGGTTCAGGAACCGTGATGAGCCACTTAAAGATGAGAA
AAACAAGCAAATGGTGACTTTCATGGATATTGTGGCATACTTGAAGCCCA
AGTATGTTCTCATGGAAAATGTCGTGGACATACTGAAATTTGCTGATGGC
TACCTAGGAAAATATGCTTTGAGCTGCCTAGTTGCTATGAAGTACCAAGC
ACGCCTTGGAATGATGGTGGCAGGCTGCTATGGTCTGCCACAGTTCAGGA
TGCGTGTGTTCCTCTGGGGTGCTCTTTCTTCCATGGTGCTCCCTAAGTAT
CCTCTGCCTACATATGACGTTGTAGTACGTGGAGGAGCACCTAATGCCTT
TTCGCAATGTATGGTTGCATATGATGAGACACAAAGGCCATCCCTGAAGA
AAGCTTTGCTTCTTGGTGATGCATTTTCAGATTTACCAAAGGTCGAAAAC
CATCAACCTAACGATGTAATGGAGTATGGTGGTTCCCCCAAGACAGAGTT
CCAGCGCTACATTCGACTTGGTCGTAAAGACATGTTGGATTGGTCGTTTG
GTGAGGAGGCTGGTCCAGATGAAGGCAAGCTCTTGGATCACCAGCCCTTA
CGGCTTAACAATGATGATTATGAGCGGGTTAAGCAAATTCCTGTCAAGAA
GGGAGCCAACTTCCGTGACCTAAAGGGTGTCAAGGTTGGAGCAAATAATG
TTGTTGAGTGGGATCCAGAAGTCGAACGTGTGTACCTTTCGTCTGGGAAA
CCACTGGTTCCTGACTATGCGATGTCATTCATCAAGGGCAAATCACTCAA
GCCATTCGGGCGCCTGTGGTGGGACGAGACGGTTCCTACAGTTGTGACCA
GAGCAGAGCCTCATAACCAGGTTATATTGCATCCGACTCAAGCAAGAGTC
TTGACTATCCGGGAGAACGCAAGGTTACAGGGCTTCCCCGATTACTACCG
ATTGTTTGGACCGATCAAGGAGAAGTAAGTTCCATCTTTTCAGTTTGCTT
GTACTTGATCTATTAACTCTTGGCAGTTGTCAGCTGTTAAGGATTGATCA
GTCAATTACAGCTCTTGAAATCAGAAGCAGTTACTGACATAAACTGCACG
GCGATGTGTTACTAACATGTGGGCCTAGGGCCCTAGGTGCTTTAGACCCA
TAGGGATCATGCTGCATTGCAATCCTTCTGAACAATGTGTGCTTTCTCAT
GGCTATAGGTATATTCAAGTCGGGAACGCAGTGGCAGTCCCTGTTGCACG
GGCACTGGGCTACTGTCTGGGTCAAGCCTACCTGGGTGAATCTGACGGGA
GTCAGCCTCTGTACCAGCTGCCTGCAAGTTTTACCTCTGTGGGGCGAACC
GCGGTTCAGGCGAATGCCGTTTCTGTTGGCACTCCTGCGGGGGAGGTAGT
CGAGCAGTAAAAGGATAGCGGAGCAACCCTGGTTGGTATTTTGATTCGAG
CCCATCCAGTAGCATGTTTACCAATAAATAATCATTGGTCGTGCTGATTC
TTATGGTTGGAGATGAATGTATGTAGGGTGTACTCGAGCTCGAGTGCTTG
TTGTACTGTAGGTTGAGGTTTCTCATCCATTGGCCTGCCTATTTGTGGAT
GACGTTTCATTTCAGATTAGCAATGTGCTTATTTAA (SEQ ID NO: 3
7)

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An F₁ hybrid maize seed, comprising
an expression cassette comprising a maize promoter that is active during meiosis and operably linked to a nucleic acid encoding an artificial microRNA (amiRNA) that targets ZMET5 (SEQ ID NO: 37).

2. An F₁ hybrid maize seed produced by crossing two different maize inbred lines, one of which comprises an expression cassette that includes a maize promoter that is active during meiosis and is operably linked to a nucleic acid encoding an artificial microRNA (amiRNA) that targets ZMET5 (SEQ ID NO: 37).

3. (canceled)

4. The F₁ hybrid maize seed of claim 1, wherein the maize promoter is a AMEIOTIC1 promoter, a AFD1/REC8 promoter, a DMC1 promoter, or a PHS1 promoter.

5. The F₁ hybrid maize seed of claim 4, wherein the amiRNA has a length of 15-30 nucleotides.

6-8. (canceled)

9. The F₁ hybrid maize seed of claim 1, wherein the amiRNA is encoded by a nucleic acid comprising at least one nucleic acid sequence selected from SEQ ID NO: 11 - SEQ ID NO: 20.

10-11. (canceled)

12. An F₁ hybrid maize plant produced by growing the F₁ hybrid maize seed of claim 1.

13. A BC₁ maize seed produced by crossing the F₁ hybrid maize plant of claim 12 to a parental line or other line.

14. An F₂ maize seed produced by self-pollinating the F₁ hybrid maize plant of claim 12.

15. An F₂ maize plant produced by growing the F₂ maize seed of claim 13.

16. An F₁ hybrid maize plant, comprising
an expression cassette comprising a maize promoter that is active during meiosis and operably linked to a nucleic acid encoding an artificial microRNA (amiRNA) that targets ZMET5 (SEQ ID NO: 37).

17. (canceled)

18. The F₁ hybrid maize plant of claim 16, wherein the maize promoter is a AMEIOTIC1 promoter, a AFD1/REC8 promoter, a DMC1 promoter, or a PHS1 promoter.

19. The F₁ hybrid maize plant of claim 16, wherein the amiRNA is encoded by a nucleic acid comprising at least one nucleic acid sequence selected from SEQ ID NO: 11 - SEQ ID NO: 20.

20. A method, comprising:
(a) introducing into an embryo of a maize plant or a cell of a maize plant an expression cassette that includes a maize promoter that is active during meiosis and is operably linked to a nucleic acid encoding an artificial microRNA (amiRNA) that targets ZMET5 (SEQ ID NO: 37) to produce an embryo comprising the expression cassette or a cell comprising the expression cassette;
(b) regenerating the embryo comprising the expression cassette or the cell comprising the expression cassette of (a) to produce a T₀ maize plant; and
(c) crossing the T₀ maize plant of (b) to a maize plant to produce F₁ maize seed.

21. The method of claim 20 further comprising growing the F₁ maize seed to produce an F₁ maize plant.

22. The method of claim 21 further comprising crossing the F₁ maize plant to a parental line to produce BC₁ maize seed, or self-pollinating the F₁ maize plant to produce F₂ maize seed.

23. The method of claim 22 further comprising growing the BC₁ maize seed to produce a BC₁ maize plant, or growing the F₂ maize seed to produce an F₂ maize plant.

24. The method of claim 23 further comprising screening the BC₁ maize plant or the F₂ maize plant for at least one desirable trait.

25. A progeny maize seed produced by a subsequent generation of the F₁ hybrid maize plant of claim 16.

26. The progeny maize seed of claim 25, wherein subsequent generation is produced by self-pollinating an F₁ hybrid maize plant or by backcrossing an F₁ hybrid maize plant to a parental line or other line.

27. The method of claim 20, wherein the amiRNA has a length of 15-30 nucleotides.

28. The method of claim 20, wherein the amiRNA is encoded by a nucleic acid comprising at least one nucleic acid sequence selected from SEQ ID NO: 11 - SEQ ID NO: 20.

29. The method of claim 20, wherein the embryo is an immature embryo or a mature embryo, and wherein the cell is from a nodal region, a leaf tissue, an anther, a tassel and ear meristem, or a shoot meristem.


SEQUENCE LISTING (37 sequences)

SEQ ID NO: 1 - DNA, 21 bp, synthetic construct
  misc_feature 1..21: note = Synthetic Polynucleotide
  source 1..21: mol_type = other DNA
tttctgtaccgattaaacccg

SEQ ID NO: 2 - DNA, 21 bp, synthetic construct
  misc_feature 1..21: note = Synthetic Polynucleotide
  source 1..21: mol_type = other DNA
tttctgtaccgataaaacccg

SEQ ID NO: 3 - DNA, 21 bp, synthetic construct
  misc_feature 1..21: note = Synthetic Polynucleotide
  source 1..21: mol_type = other DNA
ttaaatgttacctggtgactg

SEQ ID NO: 4 - DNA, 21 bp, synthetic construct
  misc_feature 1..21: note = Synthetic Polynucleotide
  source 1..21: mol_type = other DNA
ttttagattagataccgtcac

SEQ ID NO: 5 - DNA, 21 bp, synthetic construct
  misc_feature 1..21: note = Synthetic Polynucleotide
  source 1..21: mol_type = other DNA
ttttttaacggactcggtcaa

SEQ ID NO: 6 - DNA, 21 bp, synthetic construct
  misc_feature 1..21: note = Synthetic Polynucleotide
  source 1..21: mol_type = other DNA
tcatagtataggttgcgactc

SEQ ID NO: 7 - DNA, 21 bp, synthetic construct
  misc_feature 1..21: note = Synthetic Polynucleotide
  source 1..21: mol_type = other DNA
ttagtataggtcgtacctctc

SEQ ID NO: 8 - DNA, 21 bp, synthetic construct
  misc_feature 1..21: note = Synthetic Polynucleotide
  source 1..21: mol_type = other DNA
ttaacatatcccgagaagctt

SEQ ID NO: 9 - DNA, 21 bp, synthetic construct
  misc_feature 1..21: note = Synthetic Polynucleotide
  source 1..21: mol_type = other DNA
ttaaatgttacctgatgactc

SEQ ID NO: 10 - DNA, 21 bp, synthetic construct
  misc_feature 1..21: note = Synthetic Polynucleotide
  source 1..21: mol_type = other DNA
tacgaagcatcaatacggctg

SEQ ID NO: 11 - DNA, 21 bp, synthetic construct
  misc_feature 1..21: note = Synthetic Polynucleotide
  source 1..21: mol_type = other DNA
tatgttagtaacacgtggccg

SEQ ID NO: 12 - DNA, 21 bp, synthetic construct
  misc_feature 1..21: note = Synthetic Polynucleotide
  source 1..21: mol_type = other DNA
ttcaagagctgtaattgactt

SEQ ID NO: 13 - DNA, 21 bp, synthetic construct
  misc_feature 1..21: note = Synthetic Polynucleotide
  source 1..21: mol_type = other DNA
tatgttagtaacacatggccg

SEQ ID NO: 14 - DNA, 21 bp, synthetic construct
  misc_feature 1..21: note = Synthetic Polynucleotide
  source 1..21: mol_type = other DNA
ttcaagagctgtaattggctt

SEQ ID NO: 15 - DNA, 21 bp, synthetic construct
  misc_feature 1..21: note = Synthetic Polynucleotide
  source 1..21: mol_type = other DNA
tctatgggtctaaagtaccta

SEQ ID NO: 16 - DNA, 21 bp, synthetic construct
  misc_feature 1..21: note = Synthetic Polynucleotide
  source 1..21: mol_type = other DNA
tctatgggtctaaagcgccta

SEQ ID NO: 17 - DNA, 21 bp, synthetic construct
  misc_feature 1..21: note = Synthetic Polynucleotide
  source 1..21: mol_type = other DNA
tttaacagctgacaattgcta

SEQ ID NO: 18 - DNA, 21 bp, synthetic construct
  misc_feature 1..21: note = Synthetic Polynucleotide
  source 1..21: mol_type = other DNA
tttaacagctgacaaccgcta

SEQ ID NO: 19 - DNA, 21 bp, synthetic construct
  misc_feature 1..21: note = Synthetic Polynucleotide
  source 1..21: mol_type = other DNA
tttaacagctgacaactccga

SEQ ID NO: 20 - DNA, 21 bp, synthetic construct
  misc_feature 1..21: note = Synthetic Polynucleotide
  source 1..21: mol_type = other DNA
ttgttagtaacacagcgccgt

SEQ ID NO: 21 - DNA, 21 bp, synthetic construct
  misc_feature 1..21: note = Synthetic Polynucleotide
  source 1..21: mol_type = other DNA
ttaatcgaagattgtccgcaa

SEQ ID NO: 22 - DNA, 21 bp, synthetic construct
  misc_feature 1..21: note = Synthetic Polynucleotide
  source 1..21: mol_type = other DNA
ttaatcgaagattgttggcaa

SEQ ID NO: 23 - DNA, 21 bp, synthetic construct
  misc_feature 1..21: note = Synthetic Polynucleotide
  source 1..21: mol_type = other DNA
tattgtcggcaaaatatgctg

SEQ ID NO: 24 - DNA, 21 bp, synthetic construct
  misc_feature 1..21: note = Synthetic Polynucleotide
  source 1..21: mol_type = other DNA
tagttaatcgaagactgtcgg

SEQ ID NO: 25 - DNA, 21 bp, synthetic construct
  misc_feature 1..21: note = Synthetic Polynucleotide
  source 1..21: mol_type = other DNA
tagttaatcgaagattctcgg

SEQ ID NO: 26 - DNA, 21 bp, synthetic construct
  misc_feature 1..21: note = Synthetic Polynucleotide
  source 1..21: mol_type = other DNA
tattgtcggcaaaatacgcta

SEQ ID NO: 27 - DNA, 21 bp, synthetic construct
  misc_feature 1..21: note = Synthetic Polynucleotide
  source 1..21: mol_type = other DNA
ttacaatataagctgtctctt

SEQ ID NO: 28 - DNA, 21 bp, synthetic construct
  misc_feature 1..21: note = Synthetic Polynucleotide
  source 1..21: mol_type = other DNA
ttacaatataagctctttcct

SEQ ID NO: 29 - DNA, 21 bp, synthetic construct
  misc_feature 1..21: note = Synthetic Polynucleotide
  source 1..21: mol_type = other DNA
ttagagtgagcgtctatccaa

SEQ ID NO: 30 - DNA, 21 bp, synthetic construct
  misc_feature 1..21: note = Synthetic Polynucleotide
  source 1..21: mol_type = other DNA
tattgtcggcaaaatatgcta

SEQ ID NO: 31 - DNA, 300 bp, synthetic construct
  misc_feature 1..300: note = Synthetic Polynucleotide
  source 1..300: mol_type = other DNA
cgatgagtttcttgccctccttaaggaatgggcagttctatgcaaaaaatatgtccaagatgtggattcaaatttagcaagctcagaggatcaagcggatgaagacagccctcttgacaaggacgaatttgttgtagagaagcttgtcgggatatgttatggtggcagtgacagggaaaatggcatctattttaaggtccagtgggaaggatacggccctgaggaggatacatgggaaccgattgataacttgagtgactgcccgcagaaaattagagattttgtacaagaagggcacaa

SEQ ID NO: 32 - DNA, 300 bp, synthetic construct
  misc_feature 1..300: note = Synthetic Polynucleotide
  source 1..300: mol_type = other DNA
tgatgagtttcttgctctccttaaggaatgggcagtcttatgtgaaaaatatgttcatcaagacgtggattcgaatttagcaggttcagaggatcaagaggatgctgacactcttgacaaggatgaatttgttgtacagaagcttattggaatacgctatgatggcactggaaggaaaaaaggcgtatattttaaggtccaatgggaaggatatggtcctgaagaggatacatgggaacctattgataacctgagtgactgtccgctgaaaattagagaatttgtacaagaagggcgcaa

SEQ ID NO: 33 - DNA, 300 bp, synthetic construct
  misc_feature 1..300: note = Synthetic Polynucleotide
  source 1..300: mol_type = other DNA
aaaaattgtgaagctggcctgccactgtggtgctcctgattgccggaagcgcctctactaacctgcagtgtgtttatttctatttggatttttgaggaaacagcttatattgtaggaaaaacagtcatggtacctatgtagttgacatgctcttgatcatatatagcaaaagaggagcaatttatggggtgttcggtttaagaatgaaattagagatttagaggacatggtggtcatggtagcatattttgccgacaatcttcgattaactgatatatctggacatctggtcctgcaaca

SEQ ID NO: 34 - DNA, 336 bp, synthetic construct
  misc_feature 1..336: note = Synthetic Polynucleotide
  source 1..336: mol_type = other DNA
gatagagagttgcgacctatactatgacatgtcttactctgttgcatattctacatttgctaatatctcgtctgaaaatgggcagtcagacagtgataccgcttcgggtatttcttctgatgatgtggatctggagacgtcatctagtatgccaacgaggacagcaacccttcttgatctgtattctggctgtgggggcatgtctactggtctttgcttgggtgcagctctttctggcttgaaacttgaaactcgatgggctgttgatttcaacagttttgcgtgccaaagtttaaaatataatcatccacagactgaggtgcgaaatgagaaagc

SEQ ID NO: 35 - DNA, 2278 bp, Zea mays
  source 1..2278: mol_type = genomic DNA
atgccgtacgaaagctggtggacgccgaaggtgaattcggtggcgccggtgccgaacccgatattcatggacgcctctcccctgaacccgccgccgctggtggaggaggagcttgtgctgcgccgcagtgctcgttgcctcaataggccaacccggcccagctacgcggaacaggagcctcctaaaaaacccggcggccggggccggagcaagcggaagagggacgaggagaaaccggagctggcaccggcgcagcagggtgccaagagtccggacaggaaggactccaaggatgaggcgggtgagaagaagccgacgcccatgattgcggcgctgccggtctcttgcgctggcgttgctgctgaagacgatgcgacggggaccgggaagagcgccaagctgagggtgaaggagacactgagggcgttcaatagccactacctccactttgtgcaggaggagcagaagagagctcaggctgcgctacaggagaaacgcccatcaaagcggcctgacctgaaagcaatcaccaagatgcaagaaatgaatgccgtgctttatccagagaaaacgattggacatctaccagggatcgatgttggagatcatttctattctcgcgctgaaatggtagtgttgggcatacacagccattggctcaatggcattgacttcatggggctgaaataccaaggaaaggagtattcaaacttaactttcccactggccacctgtatcgtaatgtcgggggtatatgaagatgatctagacaaagctgatgaaattatttatactggtcaaggaggaaatgatttgcttggtaaccatcgccaaattggctctcaacagttgaaacgtgggaatttagcgttgaagaatagcagggagaatggtaatcctgtccgagttgttcgaggacatttgtcaaagaatagctacactggaaagatctacacctatgatgggctttacaaggttgtagatgactgggtgcagaagggagtgcaaggccatgttgttttcaaatttaagttgaagcggcttgagggtcagccatcattgacaacctctgaggtgcgatttactcgtgcagaagctccgacaactatttccgaattacccgggctggtttgtgatgacatctctggagggcaagaaaatattcctattcctgctactaacttggttgatgatccacctgttcctccatctggtttcaagtacttgaaatctctacaaattccaaaggacatcaagattccgtctagtattattggctgtgattgtgaaggaggttgtgctagcaacaaaaaatgtttgtgtgctcagcgcaatggttctgatctgccttatgtatcatataagaacattggcaggttggtggagccgaaagcagttgtatttgaatgtggggctaactgcagctgcaaccatgattgtgtaaacagaacatctcaacaaggtttgcagtaccgcttagaggtatttaagacagcttcaaaaggatggggagtcaggacttgggacactatcctccctggggctcccatctgtgagtacacaggggtgttgaggaggaccgaagatttggatggttcacagaacaactattgttttgacattgactgtcttcaaaccatgaagggtttggatggaagggagaaaagggctggatccgaaatgcacctgcctaatcttcatcctgagaatgattcagatgcacaacctgcacctgagtactgcatagacgctcactctattggcaactttgcaagatttataaatcacagctgccaacctaaccttttcgtccagtgcgtcttgagctcacacaatgatgttaaactggcgaaagtgatgcttttcgctgctgacaccatacttcctcttcaggagctatcctatgactacggttatcgattggacagtgttgttgggcctgatggaaaaattgtgaagctggcctgccactgtggtgctcctgattgccggaagcgcctctactaacctgcagtgtgtttatttctatttggatttttgaggaaacagcttatattgtaggaaaaacagtcatggtacctatgtagttgacatgctcttgatcatatatagcaaaagaggagcaatttatggggtgttcggtttaagaatgaaattagagatttagaggacatggtggtcatggtagcatattttgccgacaatcttcgattaactgatatatctggacatctggtcctgcaacacttatcctctggataaatatatatacgttaca

SEQ ID NO: 36 - DNA, 3809 bp, Zea mays
  source 1..3809: mol_type = genomic DNA
attcgatttctacacctccagagcactcactctcctggcaaaccccttcgtctcccaaccctagagagcagcagcagctaccgcagcccctgccatggcgccgagctccccgtcacccgccgcgcccacacgcgtctctgggcggaagcgcgccgccaaggccgaggagatccaccagaacaaggaggaggaggaggaggaggaggaggtcgtggcggcgtcctccgccaagcgcagccgcaaggcggcatcttctgggaagaagcccaagtcgccccccaagcaggccaagccggggaggaagaagaagggggatgccgagatgaaggagcccgtggaggacgacgtgtgcgccgaggagcccgacgaggaggagttggccatgggcgaggaggaggccgaggagcaggccatgcgggaggaggtggttgcggtcgcggcggggtcacccgggaagaagagggtggggagaaggaacgccgccgccgccgccggcgaccacgagccggagttcatcggcagccctgttgccgcggacgaggcgcgcagcaactggcccaagcgctacggccgcagcactgccgcaaagaaaccggatgaggaggaagagctcaaggccagatgtcactaccggagcgctaaggtggacaacgtcgtctactgccttggggatgacgtctatgtcaaggctggagaaaacgaggcagattacattggccgcattactgaattttttgaggggactgaccagtgtcactattttacttgccgttggttcttccgagcagaggacacggttatcaattctttggtgtccataagtgtggatggccacaagcatgaccctagacgtgtttttctttctgaggaaaagaacgacaatgtgcttgattgcattatctccaaggtcaagatagtccatgttgatccaaatatggatccaaaagccaaggctcagctgatagagagttgcgacctatactatgacatgtcttactctgttgcatattctacatttgctaatatctcgtctgaaaatgggcagtcagacagtgataccgcttcgggtatttcttctgatgatgtggatctggagacgtcatctagtatgccaacgaggacagcaacccttcttgatctgtattctggctgtgggggcatgtctactggtctttgcttgggtgcagctctttctggcttgaaacttgaaactcgatgggctgttgatttcaacagttttgcgtgccaaagtttaaaatataatcatccacagactgaggtgcgaaatgagaaagccgatgagtttcttgccctccttaaggaatgggcagttctatgcaaaaaatatgtccaagatgtggattcaaatttagcaagctcagaggatcaagcggatgaagacagccctcttgacaaggacgaatttgttgtagagaagcttgtcgggatatgttatggtggcagtgacagggaaaatggcatctattttaaggtccagtgggaaggatacggccctgaggaggatacatgggaaccgattgataacttgagtgactgcccgcagaaaattagagattttgtacaagaagggcacaaaagaaagattctcccactgcctggtgatgttgatgtcatttgtggaggcccaccatgccaaggtatcagtgggtttaatcggtacagaaaccgtgatgagccactcaaagatgagaaaaacaaacaaatggtgactttcatggatattgtggcgtacttgaagcccaagtatgttctcatggaaaatgtggtggacatactcaaatttgcggatggttacctaggaaaatatgctttgagctgccttgttgctatgaagtaccaagcgcggcttggaatgatggtggctggttgctatggtctgccacagttcaggatgcgtgtgttcctctggggtgctctttcttccatggtgctccctaagtatcctctgcccacctatgatgttgtagtacgtggaggagcccctaatgccttttcgcaatgtatggttgcatatgacgagacacaaaaaccatccctgaaaaaagccttgcttcttggcgatgcaatttcagatttaccaaaggttcaaaatcaccagcctaacgatgtgatggagtatggtggttcccccaagaccgaattccagcgctacattcgactcagtcgtaaagacatgttggattggtccttcggtgagggggctggtccagatgaaggcaagctcttggatcaccagcctttacggcttaacaacgatgattatgagcgggttcaacagattcctgtcaagaagggagccaacttccgcgacctaaagggcgtgagggttggagcaaacaatattgttgagtgggatccagaaatcgagcgtgtgaaactttcatctgggaaaccactggttcctgactatgcaatgtcattcatcaagggcaaatcactcaagccgtttgggcgcctgtggtgggacgagacagttcctacagttgtaaccagagcagagcctcacaaccaggttataattcatccgactcaagcaagggtcctcactatccgggagaacgcaaggttacagggcttccccgattactaccgattgtttggcccgatcaaggagaagtacattcaagtcgggaacgcagtggctgtccctgttgcccgggcactgggctactgtctggggcaagcctacctgggtgaatctgaggggagtgaccctctgtaccagctgcctccaagtttcacctctgttggaggacgcactgcggggcaggcgagggcctctcctgttggcacccctgcaggggaggtagttgagcagtaaaaggatgacagatctgagctgagctgggcaacatccagcggcaggagcatttctggttcggttcgattcgggctcacgagcgcattgtgctgattcgcagggtgcactcgagctcgagtgcggagttgttgtaatgtaggttgaggtttcaaaacatccatccatccatcggccccgcctatttgtggatggtgtttctcgtgccattttagttcagctgtattgatgcttcgtcatgtatgtgtattgtaccattcgttcttgttacctactagtaaggcaacatttgatccatgttcaacgcgagcaaaattccatccactttaggtactgagatgctcaagtagttcagtcatcaggtaacatttacaacctattattttgcctggtattatctgaaaacgatttgctgaggatgttttttttttgaagtaaaggtactgtaggaaggtcctacagtaatattattaaatcaaaagaactaagtttagggagacagaagaatattacaaagaaataaaagggagagtgaggagggggaagaattctggatacatgtatttactctatttcacataaacatatggacatcaacatagatgtgaaggtatctaatctaaataagcgccgacatgctttacaagatagtttgatctataaggtgagggatgttgtggatgaagattgtgaccaactagctcatgtgtagttttgtttgtagtacctgtcctaaaaaatatgtaattctaagtttaatttgaatcagccttgttttttaaaatttttttgaccaagtccgttaaaaagtaataacattttttacttcaaatagatttactatgaaattttgtcttatggttatctaatgacatttatttaaaactgtaacaaaaactggtttggttaggggactgagggagtagtgcttaaaatggaagacgataggagagatcacattagtggtggc

SEQ ID NO: 37 - DNA, 3236 bp, Zea mays
  source 1..3236: mol_type = genomic DNA
agcagcagcagctaccgcatcccttgccatggcgccaagctccccgtcatccgctaggcccacgcgcgcctccggccggaagcgctccgccatggccgaggagatccaccagaaccaggaggaggaggaggaggttgtggctgcgtccaccgccaagcgcagacgcaaggcggcgtcttccgggaagaagcccaagccgacgcccaagcaggccaaaccggccgtggcggggatgaagaagaagggggagaccgagaagacggagcccgttgtggacgacgtgtgcgccgaggagcccgacgaggaggagttggccatgggcgaggaggaggcggaggccgaggagcaggccatgcaggaggtggtggccgcggtcgcggccgggtcgcccggtaagaagagggtggggagaaggagcgccgccgcctcgggcgaccacgtgcccgagttcatcggcagccccgttgctgcggccgaggcgcacagcaactggcccaagcgctacgagcgcagcactgccgcaaataaaccggaggaggacgatgagctcaaggccaggtgccactaccggagcgctaaggtggacaacattgtctactgcctcggggatgacgtttatgtcaaggctggagaaaatgaggctgattacattggtcgcattactgaattttttgagggcactgatcgatgccactattttacttgccgttggtttttccgtgcagaggacacggtcatcaattcgttggtgtcgattaatgttgatggccacaagcatgaccctagacgtgtttttctttccgaggaaaagaatgacaatgtgcttgattgcatcatctccaaggtcaagatagtccatgttgatccaaatatggatccaaaagctaaggcccaactgatagaacattgtgacctatactatgacatgtcttactctgttgcatattctacatttgctaatatctcatcagaaaatgggcagtcaggcagtgaaaccgcttcgggtatttcttctgatgatgcgggtttggagacgtcatctaatatgccagagaggacagcaacccttcttgacttgtattcgggctgtgggggcatgtccactggtctttgcttgggtgctgctctttctggcctgaaacttgaaactcgatgggctgttgatcttaacagttttgcgtgccaaagtttaaaatacaatcatccacagactgaggtgcgaaatgagaaagctgatgagtttcttgctctccttaaggaatgggcagtcttatgtgaaaaatatgttcatcaagacgtggattcgaatttagcaggttcagaggatcaagaggatgctgacactcttgacaaggatgaatttgttgtacagaagcttattggaatacgctatgatggcactggaaggaaaaaaggcgtatattttaaggtccaatgggaaggatatggtcctgaagaggatacatgggaacctattgataacctgagtgactgtccgctgaaaattagagaatttgtacaagaagggcgcaaaagaaaaattctcccgcttcctggtgatgttgatgtcatttgtggaggcccaccatgtcaaggtataagtgggtttaatcggttcaggaaccgtgatgagccacttaaagatgagaaaaacaagcaaatggtgactttcatggatattgtggcatacttgaagcccaagtatgttctcatggaaaatgtcgtggacatactgaaatttgctgatggctacctaggaaaatatgctttgagctgcctagttgctatgaagtaccaagcacgccttggaatgatggtggcaggctgctatggtctgccacagttcaggatgcgtgtgttcctctggggtgctctttcttccatggtgctccctaagtatcctctgcctacatatgacgttgtagtacgtggaggagcacctaatgccttttcgcaatgtatggttgcatatgatgagacacaaaggccatccctgaagaaagctttgcttcttggtgatgcattttcagatttaccaaaggtcgaaaaccatcaacctaacgatgtaatggagtatggtggttcccccaagacagagttccagcgctacattcgacttggtcgtaaagacatgttggattggtcgtttggtgaggaggctggtccagatgaaggcaagctcttggatcaccagcccttacggcttaacaatgatgattatgagcgggttaagcaaattcctgtcaagaagggagccaacttccgtgacctaaagggtgtcaaggttggagcaaataatgttgttgagtgggatccagaagtcgaacgtgtgtacctttcgtctgggaaaccactggttcctgactatgcgatgtcattcatcaagggcaaatcactcaagccattcgggcgcctgtggtgggacgagacggttcctacagttgtgaccagagcagagcctcataaccaggttatattgcatccgactcaagcaagagtcttgactatccgggagaacgcaaggttacagggcttccccgattactaccgattgtttggaccgatcaaggagaagtaagttccatcttttcagtttgcttgtacttgatctattaactcttggcagttgtcagctgttaaggattgatcagtcaattacagctcttgaaatcagaagcagttactgacataaactgcacggcgatgtgttactaacatgtgggcctagggccctaggtgctttagacccatagggatcatgctgcattgcaatccttctgaacaatgtgtgctttctcatggctataggtatattcaagtcgggaacgcagtggcagtccctgttgcacgggcactgggctactgtctgggtcaagcctacctgggtgaatctgacgggagtcagcctctgtaccagctgcctgcaagttttacctctgtggggcgaaccgcggttcaggcgaatgccgtttctgttggcactcctgcgggggaggtagtcgagcagtaaaaggatagcggagcaaccctggttggtattttgattcgagcccatccagtagcatgtttaccaataaataatcattggtcgtgctgattcttatggttggagatgaatgtatgtagggtgtactcgagctcgagtgcttgttgtactgtaggttgaggtttctcatccattggcctgcctatttgtggatgacgtttcatttcagattagcaatgtgcttatttaa